(19) United States
(12) Patent Application Publication
(10) Pub. No.: US 2010/0137309 A1
(43) Pub. Date: Jun. 3, 2010

(54) FUNGICIDES PHENYL-PYRIMIDNYL-AMINO DERIVATIVES

(76) Inventors: Pierre-Yves Coqueron, Lyon (FR); Ralf Ralf Dunkel, Lyon (FR); Pierre Genix, Lyon (FR); Jorg Greul, Leichlingen (DE); Marie-Claire Grosjean-Cournoyer, Curis Mont D'or (FR)

Correspondence Address: OSTROLENK FABER GERB & SOFFEN, 1180 AVENUE OF THE AMERICAS, NEW YORK, NY, 100368403, US

(21) Appl. No.: 12/451,440

(22) PCT Filed: May 16, 2008
(86) PCT No.: PCT/EP2008/056072

§ 371 Date: Jan. 28, 2010

(30) Foreign Application Priority Data
May 16, 2007 (EP) 07356067.4

(51) Int. Cl. A01N 43/54; C07D 401/14; C07D 401/04; C07D 413/14; A01N 43/84; A01P 3/00

(52) U.S. Cl. 514/235.8; 544/331; 544/122; 544/295; 514/275

(57) ABSTRACT

The present invention relates to phenyl-pyrimidinyl-amino derivatives of formula (I) wherein Q¹ and p, R^{a} to R^{c}, L¹, Y, L² and Q² represent various substituents, their process of preparation, preparation intermediate compounds, their use as fungicide active agents, particularly in the form of fungicide compositions, and methods for the control of phytopathogenic fungi, notably of plants, using these compounds or compositions.

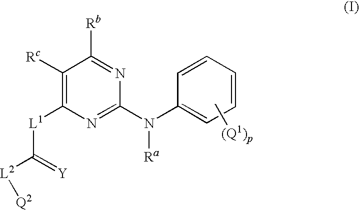

The present invention relates to phenyl-pyrimidinyl-amino derivatives, their process of preparation, preparation intermediate compounds, their use as fungicide active agents, particularly in the form of fungicide compositions, and methods for the control of phytopathogenic fungi, notably of plants, using these compounds or compositions.

In international patent application WO-2003/049542, there are pyrazolyl-pyrimidinyl-amino derivatives described that present a chemical structure having some similarities with the compounds according to the invention. However, the said chemical structure of these compounds of the prior art is different from the compounds of the present invention.

Apart, certain phenyl-pyrimidinyl-amino derivatives have been disclosed in the prior art but do not possess any or sufficient biological efficacies hence do not form part of the present invention. Such compounds have been excluded from the scope of the present invention.

It is always of high-interest in agriculture to use novel pesticide compounds in order to avoid or to control the development of resistant strains to the active ingredients. It is also of high-interest to use novel compounds being more active than those already known, with the aim of decreasing the amounts of active compound to be used, whilst at the same time maintaining effectiveness at least equivalent to the already known compounds. We have now found a new family of compounds which possess the above mentioned effects or advantages.

Accordingly, the present invention provides phenyl-pyrimidinyl-amino derivatives of formula (I)

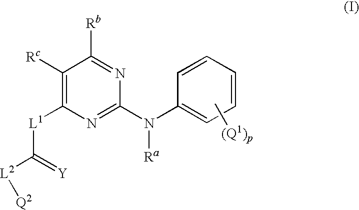

wherein
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylsulfanyl, substituted or non-substituted benzylamino, substituted or non-substituted phenoxy, substituted or non-substituted phenylsulfanyl, or substituted or non-substituted phenylamino ;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, a C₁-C₈-halogenoalkoxyalkyl having 1 to 5 halogen atoms,
R^{b} and R^{c} independently represent a hydrogen atom, a halogen atom, a cyano, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms ;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₈-alkyl, substituted or non-substituted (C₁-C₈-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₈-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylsulfanyl, substituted or non-substituted benzylamino, substituted or non-substituted phenoxy, substituted or non-substituted phenylsulfanyl, substituted or non-substituted phenylamino, a substituted or non-substituted or a 4-, 5-, 6- or 7-membered heterocycle comprising up to 4 heteroatoms selected in the list consisting of N, O, S;
L² and Q² can form together a substituted or non-substituted, 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, substituted or non-substituted (hydroxyimino)-C₁-C₆-alkyl group, substituted or non-substituted C₁-C₈-alkyl, tri(C₁-C₈-alkyl)silyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, or a 4-, 5-, 6- or 7-membered heterocycle comprising up to 4 heteroatoms selected in the list consisting of N, O, S ; as well as salts, N-oxides, metallic complexes, metalloidic complexes and optically active or geometric isomers thereof; provided that the following compounds are excluded:
5-(2-{[3-(trifluoromethyl)phenyl]amino}pyrimidin-4-yl)nicotinamide;
5-{2-[(tert-butoxycarbonyl)(3-chlorophenyl)amino]-6-methylpyrimidin-4-yl}nicotinic acid;
5-{2-[(tert-butoxycarbonyl)(3-chlorophenyl)amino]pyrimidin-4-yl}nicotinic acid;
ethyl 5-{2-[(3-chlorophenyl)amino]-6-methylpyrimidin-4-yl}nicotinate;
ethyl 5-{2-[(tert-butoxycarbonyl)(3-chlorophenyl)amino]-6-methylpyrimidin-4-yl}nicotinate;
4-(2-{[3-(1,1,2,2-tetrafluoroethoxy)phenyl]amino}pyrimidin-4-yl)pyridine-2-carboxamide;
4-(2-{[3-(1,1,2,2-tetrafluoroethoxy)phenyl]amino}pyrimidin-4-yl)pyridine-2-carboxylic acid;
N-(2-hydroxyethyl)-4-(2-{[3-(1,1,2,2-tetrafluoroethoxy)-phenyl]amino}pyrimidin-4-yl)pyridine-2-carboxamide;
N-(3-hydroxypropyl)-4-(2-{[3-(1,1,2,2-tetrafluoroethoxy)-phenyl]amino}pyrimidin-4-yl)pyridine-2-carboxamide;
N-(2-aminoethyl)-4-(2-{[3-(1,1,2,2-tetrafluoroethoxy)-phenyl]amino}pyrimidin-4-yl)pyridine-2-carboxamide hydrochloride;
N-(3-aminopropyl)-4-(2-{[3-(1,1,2,2-tetrafluoroethoxy)-phenyl]amino}pyrimidin-4-yl)pyridine-2-carboxamide hydrochloride;
N-(2-aminoethyl)-4-(2-{[3-(1,1,2,2-tetrafluoroethoxy)-phenyl]amino}pyrimidin-4-yl)pyridine-2-carboxamide;
N-(3-aminopropyl)-4-(2-{[3-(1,1,2,2-tetrafluoroethoxy)-phenyl]amino}pyrimidin-4-yl)pyridine-2-carboxamide;
4-{2-[(3-chlorophenyl)amino]pyrimidin-4-yl}pyridine-2-carboxylic acid;
4-(2-{[3-(trifluoromethyl)phenyl]amino}pyrimidin-4-yl)pyridine-2-carboxylic acid;
4-{2-[(3-chlorophenyl)amino]pyrimidin-4-yl}pyridine-2-carboxamide;
4-(2-{[3-(trifluoromethyl)phenyl]amino}pyrimidin-4-yl)pyridine-2-carboxamide;
4-{2-[(3-chlorophenyl)amino]pyrimidin-4-yl}pyridine-2-carboxylic acid;
N-(2-aminoethyl)-4-{2-[(3-chlorophenyl)amino]pyrimidin-4-yl}pyridine-2-carboxamide hydrochloride;
4-{2-[(3-chlorophenyl)amino]pyrimidin-4-yl}-N-(2-hydroxyethyl)-pyridine-2-carboxamide;
4-{2-[(3-chlorophenyl)amino]pyrimidin-4-yl}-N-(3-hydroxypropyl)-pyridine-2-carboxamide;
N-(3-aminopropyl)-4-{2-[(3-chlorophenyl)amino]pyrimidin-4-yl}pyridine-2-carboxamide hydrochloride;
4-{2-[(3-chlorophenyl)amino]pyrimidin-4-yl}pyridine-2-carboxylic acid-monosodium salt.

Any of the compounds according to the present invention may exist in one or more optical or chiral isomeric form depending on the number of asymmetric centres in the compound. The invention thus relates equally to all optical isomers and to any racemic or scalemic mixtures thereof (the term “scalemic” denotes a mixture of enantiomers in different proportions), and to the mixtures of any potential stereoisomers, in any proportion. Diastereoisomers or optical isomers can be separated according to any methods known per se by the man ordinary skilled in the art.

Any of the compounds according to the present invention may also exist in one or more geometric isomeric form depending on the number of double bond within the compound. The invention thus equally relates to any geometric isomer and to any possible mixtures thereof, in any proportion. Geometric isomers can be separated according to any method known per se by the man ordinary skilled in the art.

Any compound of formula (I) according to the invention wherein L²Q² represents a hydroxy group, a sulfanyl group or an amino group can exist in a tautomeric form resulting from the shift of the proton of said hydroxy group, sulfanyl group or amino group respectively. Such tautomeric forms are also part of the present invention. Generally, any tautomeric form of a compound of formula (I) according to the invention wherein L²Q² represents a hydroxy group, a sulfanyl group or an amino group, as well as the tautomeric forms of the compounds which can optionally be used as intermediates in the preparation processes according to the invention are also part of the present invention.

According to the invention, the following generic terms are generally used with the following meanings:
halogen means fluorine, chlorine, bromine or iodine;
heteroatom can be nitrogen, oxygen or sulphur;
unless indicated otherwise, a group or a substituent that is substituted according to the invention can be substituted by one or more of the following groups or atoms: a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulfanyl, benzylamino, phenoxy, phenylsulfanyl, or phenylamino.

Preferred compounds of formula (I) according to the invention are those wherein Q¹ represents a halogen atom, a nitro group, a hydroxy group, a cyano group, an amino group, a sulfanyl group, a pentafluoro-λ⁶-sulfanyl group, a formyl group, a formyloxy group, a formylamino group, a (hydroxyimino)-C₁-C₆-alkyl group, a C₁-C₈-alkyl, a substituted or non-substituted tri(C₁-C₈-alkyl)silyl-C₁-C₈-alkyl, substituted or non-substituted C₁-C₈-cycloalkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₁-C₈-alkylcarbonyl, a C₁-C₈-halogenoalkylcarbonyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a (C₁-C₆-alkoxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms.

Other preferred compounds of formula (I) according to the invention are those wherein p represents 0, 1, 2, or 3. More preferably, p represents 0 or 1. Even more preferably p represents 1.

Other preferred compounds of formula (I) according to the invention are those wherein R^{a} represents a hydrogen atom or a C₁-C₈-cycloalkyl,

Other preferred compounds of formula (I) according to the invention are those wherein R^{b} and R^{c} independently represent a hydrogen atom, a halogen atom, a cyano, a C₁-C₈-halogenoalkyl having 1 to 5 halogen atoms, a C₁-C₈-halogenocycloalkyl having 1 to 5 halogen atoms. More preferably, R^{b} and R^{c} independently represent a hydrogen atom or a halogen atom.

Other preferred compounds of formula (I) according to the invention are those wherein L¹ is selected in the list consisting of:

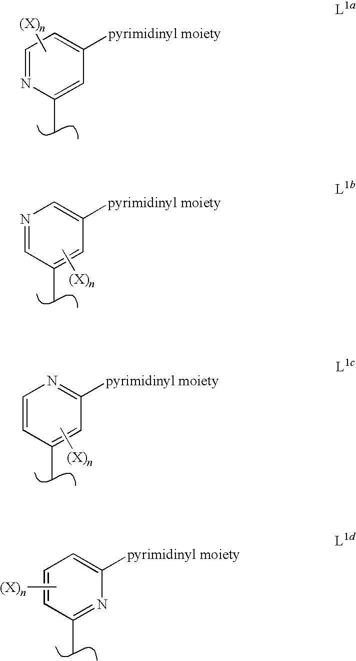

wherein
n represents 0, 1, 2 or 3;
X independently represents a C₁-C₁₀-alkyl, a C₁-C₁₀-halogenoalkyl, a halogen atom or a cyano.

Other preferred compounds of formula (I) according to the invention are those wherein Q² represents a hydrogen atom, a halogen atom, a hydroxy group, a cyano group, an amino group, a sulfanyl group, a formyl group, a formyloxy group, a formylamino group, a carbamoyl group, a N-hydroxycarbamoyl group, a carbamate group, substituted or non-substituted (hydroxyimino)-C₁-C₈-alkyl group, substituted or non-substituted C₁-C₈-alkyl, substituted or non-substituted C₁-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulfanyl, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-halogenoalkoxycarbonyl having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₈-alkyl, substituted or non-substituted (C₁-C₈-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylsulfanyl, substituted or non-substituted benzylamino, substituted or non-substituted phenoxy, substituted or non-substituted phenylsulfanyl, substituted or non-substituted phenylamino, a substituted or non-substituted or a 4-, 5-, 6- or 7-membered heterocycle comprising up to 4 heteroatoms selected in the list consisting of N, O, S. When L² and Q² form together a substituted or non-substituted, 4-, 5-, 6- or 7-membered heterocycle comprising up to 4 heteroatoms selected in the list consisting of N, O, S, preferred resulting heterocycles are non-aromatic. More preferred heterocycles are substituted or non-substituted pyrrolidine, substituted or non-substituted piperidine, substituted or non-substituted morpholine.

Other preferred compounds of formula (I) according to the invention are those wherein R^{d} to Rⁱ independently represent a hydrogen atom, a halogen atom, a nitro group, a cyano group, a hydroxy group, an amino group, a sulfanyl group, a formyl group, a formyloxy group, a formylamino group, substituted or non-substituted (hydroxyimino)-C₁-C₆-alkyl group, substituted or non-substituted C₁-C₈-alkyl, tri(C₁-C₈-alkyl)silyl, substituted or non-substituted tri(C₁-C₈-alkyl)silyl-C₁-C₈-alkyl, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, or a 4-, 5-, 6- or 7-membered heterocycle comprising up to 4 heteroatoms selected in the list consisting of N, O, S.

The above mentioned preferences with regard to the substituents of the compounds of formula (I) according to the invention can be combined in various manners, either individually, partially or entirely. These combinations of preferred features thus provide sub-classes of compounds according to the invention. Examples of such sub-classes of preferred compounds according to the invention can combine:
preferred features of Q¹ and p with preferred features of one or more of R^{a} to Rⁱ, L¹, Y, L² and Q²;
preferred features of R^{a} to Rⁱ with preferred features of one or more of Q¹ and p, L¹, Y, L² and Q²;
preferred features of L¹ with preferred features of one or more of Q¹ and p, R^{a} to Rⁱ, Y, L² and Q²;
preferred features of Y with preferred features of one or more of Q¹ and p, R^{a} to Rⁱ, L¹, L² and Q²;
preferred features of L² with preferred features of one or more of Q¹ and p, R^{a} to Rⁱ, L¹, Y and Q²;
preferred features of Q² with preferred features of one or more of Q¹ and p, R^{a} to Rⁱ, L¹, Y and L².

In these combinations of preferred features of the substituents of the compounds according to the invention, the said preferred features can also be selected among the more preferred features of each of Q¹ and p, R^{a} to Rⁱ, L¹, Y, L² and Q² so as to form most preferred subclasses of compounds according to the invention.

The preferred features of the other substituents of the compounds according to the invention can also be part of such sub-classes of preferred compounds according to the invention, notably the groups of substituents Q¹ and p, R^{a} to Rⁱ, L¹, Y, L² and Q².

The present invention also relates to a process for the preparation of compounds of formula (I). Thus according to a further aspect of the present invention, there is provided a process P1 for the preparation of a compound of formula (I) as herein-defined, as illustrated by the following reaction scheme:

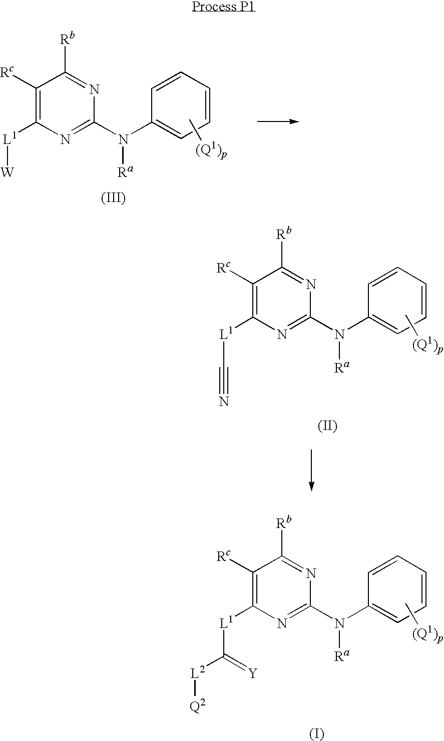

wherein
W represents a leaving group such as a halogen atom, a C₁-C₆ alkylsulfonate, a C₁-C₆ haloalkylsulfonate ; a substituted or non-substituted phenylsulfonate and
if Y represents an oxygen atom and L² represents CR^{h}Rⁱ,
Q¹, p, R^{a}, R^{b}, R^{c}, R^{h}, Rⁱ, L¹, Q² being as herein-defined; and that comprises
   reacting a compound of formula (III) with a cyanide reagent such as a metallic cyanide for example sodium cyanide, potassium cyanide, zinc cyanide; a metalloidic cyanide, an organo-metallic cyanide for example di-C₁-C₆-alkylaluminum cyanide notably di-ethylaluminum cyanide; an organo-metalloidic cyanide for example tri-C₁-C₆-alkylsilylcyanide notably tri-methylsilylcyanide in order to yield a compound of formula (II), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (II)
   then reacting said compound of formula (II) with an organo-metallic reagent of formula Q²-L²-M, wherein M represents a substituted or non-substituted metal such as lithium, magnesium, sodium, potassium or a substituted or non-substituted metallic salt such as magnesium salt, lithium salt, potassium salt or sodium salt; to yield a compound of formula (I); optionally in the presence of a catalyst;
   or by then reacting said compound of formula (II) with a phosphorane ylide reagent of formula Q²-L²-U, wherein U represents a tri-(phenyl)-phosphonium group, a di-(C₁-C₆)-alkylphosphonate; to yield a compound of formula (I); in the presence of a base, such as an inorganic or an organic base; preferably an alkaline earth metal or alkali metal hydrides, hydroxides, amides, alcoholates, acetates, carbonates or hydrogen carbonates, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU); optionally in the presence of a catalyst ;
if Y represents an oxygen atom, L² represents a direct bond and Q² represents a hydrogen atom;
Q¹, p, R^{a}, R^{b}, R^{c}, L¹, being as herein defined; and that comprises
   reacting a compound of formula (III) with a cyanide reagent such as a metallic cyanide for example sodium cyanide, potassium cyanide, zinc cyanide; a metalloidic cyanide, an organo-metallic cyanide for example di-C₁-C₆-alkylaluminum cyanide notably di-ethylaluminum cyanide; an organo-metalloidic cyanide for example tri-C₁-C₆-alkylsilylcyanide notably tri-methylsilylcyanide in order to yield a compound of formula (II), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (II),
   reacting said compound of formula (II) with a reducing agent such as hydrogen, a metal, such as magnesium, a metallic salt such as SnCl₂ or SnBr₂; or a hydride donor of formula H-M, wherein M represents a substituted or non-substituted metal, or a substituted or non-substituted metallic salt, such as di-C₁-C₆-alkylaluminum hydrides, notably di-ethylaluminum hydride, to yield a compound of formula (I); optionally in the presence of a catalyst ;
if Y represents an oxygen atom, L² represents an oxygen atom and Q² represents a hydrogen atom;
Q¹, p, R^{a}, R^{b}, R^{c}, L¹, being as herein defined; and that comprises
   reacting a compound of formula (III) with a cyanide reagent such as a metallic cyanide for example sodium cyanide, potassium cyanide, zinc cyanide; a metalloidic cyanide, an organo-metallic cyanide for example di-C₁-C₆-alkylaluminum cyanide notably di-ethylaluminum cyanide; an organo-metalloidic cyanide for example tri-C₁-C₆-alkylsilylcyanide notably tri-methylsilylcyanide in order to yield a compound of formula (II), optionally in the presence of a catalyst, preferably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (II),
   hydrolyzing said compound of formula (II), preferably in presence of water, optionally in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU); optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, or an organic acid, such as para-toluenesulphonic acid; to yield a compound of formula (I);
if Y represents NH, L² represents a direct bond, a sulphur atom, an oxygen atom or NH, and Q² represents a hydrogen atom, a halogen atom, a nitro group, a hydroxy group, a cyano group, an amino group, a sulfanyl group, a formyloxy group, a formylamino group, a carbamate group, substituted or non-substituted (hydroxyimino)-C₁-C₈-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₈-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylamino, substituted or non-substituted phenoxy or substituted or non-substituted phenylamino;
Q¹, p, R^{a}, R^{b}, R^{c}, L¹, being as herein defined; and that comprises
   reacting a compound of formula (III) with a cyanide reagent such as a metallic cyanide for example sodium cyanide, potassium cyanide, zinc cyanide; a metalloidic cyanide, an organo-metallic cyanide for example di-C₁-C₈-alkylaluminum cyanide notably di-ethylaluminum cyanide; an organo-metalloidic cyanide for example tri-C₁-C₆-alkylsilylcyanide notably tri-methylsilylcyanide in order to yield a compound of formula (II), optionally in the presence of a catalyst, notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; to yield a compound of formula (II),
   performing the addition reaction of a compound of formula (II) with a reagent of formula Q²-L²-H, optionally in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU); optionally in the presence of an acid such as a Lewis acid; notably metal or metalloid halides such as aluminium trichloride, zinc dichloride, magnesium bromide boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid; optionally in the presence of a catalyst, to yield a compound of formula (I).

Advantageously, process P1 according to the invention can be simplified, allowing the direct preparation of certain compounds of formula (I) starting from a compound of formula (III). Accordingly, the present invention provides an improved process P1A for the preparation of a compound of formula (I), as illustrated by the following reaction scheme:

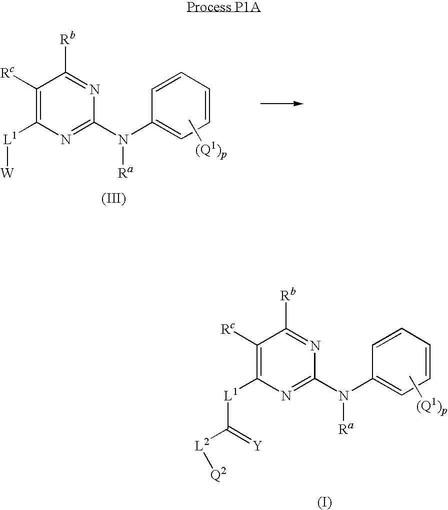

wherein
W represents a leaving group such as a halogen atom, a C₁-C₆ alkylsulfonate, a C₁-C₆ haloalkylsulfonate; a substituted or non-substituted phenylsulfonate and
if Y represents an oxygen atom, L² represents an oxygen atom, a NR^{g} group and R^{g} represents a hydrogen atom, a nitro group, a cyano group, a hydroxy group, an amino group, a formyloxy group, a formylamino group, substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, or a 4-, 5-, 6- or 7-membered heterocycle comprising up to 4 heteroatoms selected in the list consisting of N, O, S;
Q¹, p, R^{a}, R^{b}, R^{c}, L¹, Q² being as herein-defined; and that comprises
   reacting a compound of formula (III) with a compound of formula Q²-L²-H, wherein Q² is herein-defined and L² represents oxygen atom, a NR^{g} group and R^{g} represents a hydrogen atom, a nitro group, a cyano group, a hydroxy group, an amino group, a formyloxy group, a formylamino group, substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, or a 4-, 5-, 6- or 7-membered heterocycle comprising up to 4 heteroatoms selected in the list consisting of N, O, S; in the presence of carbon monoxide or a carbon monoxide generating agent such as Mo(CO)₆ or W(CO)₆, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine, optionally in the presence of a base such as an inorganic or an organic base; preferably an alkaline earth metal or alkali metal hydride, hydroxide, amide, alcoholate, amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), to yield a compound of formula (I).

The process according to the invention also allows the preparation of compounds of formula (I) according to the invention using other compounds of formula (I) according to the invention as starting material.

Thus according to a further aspect of the present invention, there is provided a process P2 for the preparation of a compound of formula (I) wherein Y represents a NR^{d} group, L² represents CR^{h}Rⁱ; Q¹, p, R^{a}, R^{b}, R^{c}, R^{d}, R^{h}, Rⁱ, L¹, Q² being as herein defined;

that comprises reacting a different compound of formula (I) wherein Y represents an oxygen atom and L² represents CR^{h}Rⁱ; Q¹, p, R^{a}, R^{b}, R^{c}, R^{h}, Rⁱ, L¹, Q² being as herein-defined;
with a compound of formula R^{d}NH or one of its salts, wherein R^{d} is as herein-defined, optionally in the presence of a dehydrating agent such as molecular sieves, anhydrous metal salts, such as magnesium sulphate, sodium sulphate, or metal oxides such as barium oxide, calcium oxide, optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P3 for the preparation of a compound of formula (I) wherein Y represents a CR^{e}R^{f} group, L² represents an oxygen atom and Q² represents a formyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, a substituted or non-substituted or a 4-, 5-, 6- or 7-membered heterocycle comprising up to 4 heteroatoms selected in the list consisting of N, O, S; Q¹, p, R^{a}, R^{b}, R^{c}, R^{h}, Rⁱ, L¹, being as herein defined;

that comprises reacting a different compound of formula (I) wherein Y represents an oxygen atom, L² represents CR^{h}Rⁱ and Q² represents a hydrogen atom; Q¹, p, R^{a}, R^{b}, R^{c}, R^{h}, Rⁱ, L¹, being as herein-defined;
with a compound of formula Q²W wherein W represents a leaving group such as a halogen atom, a C₁-C₆ alkylsulfonate, a C₁-C₆ haloalkylsulfonate and Q² represents a formyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, a substituted or non-substituted or a 4-, 5-, 6- or 7-membered heterocycle comprising up to 4 heteroatoms selected in the list consisting of N, O, S; optionally in the presence of a base such as an inorganic or an organic base; preferably an alkaline earth metal or alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amine, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU).

According to a further aspect of the present invention, there is provided a process P4 for the preparation of a compound of formula (I) wherein Y represents a NR^{d} group, L² represents a direct bond and Q² represents a hydrogen atom; Q¹, p, R^{a}, R^{b}, R^{c}, R^{d}, L¹ being as herein defined,

and that comprises reacting a different compound of formula (I) wherein Y represents an oxygen atom, L² represents a direct bond and Q² represents a hydrogen atom; Q¹, p, R^{a}, R^{b}, R^{c}, L¹ being as herein-defined;
with a compound of formula R^{d}NH or one of its salts, R^{d} being as herein-defined, optionally in the presence of dehydrating agent such as molecular sieves, anhydrous metal salts, such as magnesium sulphate, sodium sulphate, or metal oxides such as barium oxide, calcium oxide, optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P5 for the preparation of a compound of formula (I) wherein Y represents a CR^{e}R^{f} group, L² represents a direct bond and Q² represents a hydrogen atom; Q¹, p, R^{a}, R^{b}, R^{c}, L¹ being as herein defined and that comprises reacting a different compound of formula (I) wherein Y represents an oxygen atom, L² represents a direct bond and Q² represents a hydrogen atom; Q¹, p, R^{a}, R^{b}, R^{c}, L¹ being as herein-defined;

with a compound of formula CHUR^{e}R^{f}, wherein U represents a hydrogen atom, a phosphonium group, a di-(C₁-C₆)-alkylphosphonate, R^{e}, R^{f} being as herein-defined, or one of its salts, a tri-(C₁-C₆)-alkylsilyl, optionally in the presence of a base such as an inorganic or an organic base;
notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P6 for the preparation of a compound of formula (I) wherein Y represents a CR^{e}R^{f} group, L² represents CR^{h}Rⁱ, and Q¹, p, R^{a}, R^{b}, R^{c}, R^{e}, R^{f}, R^{h}, Rⁱ, L¹, Q² being as herein-defined;

and that comprises reacting a different compound of formula (I) wherein Y represents an oxygen atom and, L² represents CR^{h}Rⁱ and Q¹, p, R^{a}, R^{b}, R^{c}, R^{e}, R^{f}, R^{h}, Rⁱ, L¹, Q² being as herein-defined;
with a compound of formula CHUR^{e}R^{f} wherein U represents a hydrogen atom, a tri-(phenyl)-phosphonium group, a di-(C₁-C₆)-alkylphosphonate, R^{e}, R^{f} being as herein-defined, or one of its salts, a tri-(C₁-C₆)-alkylsilyl, optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid.

According to a further aspect of the present invention, there is provided a process P7 for the preparation of a compound of formula (I) wherein Y represents a NR^{d} group, L² represents an oxygen atom a sulphur atom or a NR^{g} group wherein R^{g} represents a hydrogen atom, a formyl group, a formyloxy group, a formylamino group, a carbamoyl group, substituted or non-substituted (hydroxyimino)-C₁-C₈-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms or a 4-, 5-, 6- or 7-membered heterocycle comprising up to 4 heteroatoms selected in the list consisting of N, O, S; Q² represents a formyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, a substituted or non-substituted or a 4-, 5-, 6- or 7-membered heterocycle comprising up to 4 heteroatoms selected in the list consisting of N, O, S; and Q¹, p, R^{a}, R^{b}, R^{d}, L¹ being as herein-defined;

and that comprises reacting a different compound of formula (I) wherein Y represents a NR^{d} group, L² represents an oxygen atom, a sulphur atom or a NR^{g} group wherein R^{g} represents a hydrogen atom, a formyl group, a formyloxy group, a formylamino group, a carbamoyl group, substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms or a 4-, 5-, 6- or 7-membered heterocycle comprising up to 4 heteroatoms selected in the list consisting of N, O, S; Q² represents a hydrogen atom; Q¹, p, R^{a}, R^{b}, R^{c}, R^{d}, L¹, being as herein-defined; with a compound of formula Q²W wherein W represents a leaving group such as a halogen atom, a C₁-C₆ alkylsulfonate, a C₁-C₆ haloalkylsulfonate and Q² represents a formyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, a substituted or non-substituted or a 4-, 5-, 6-, or 7-membered heterocycle comprising up to 4 heteroatoms selected in the list consisting of N, O, S; optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine.

According to a further aspect of the present invention, there is provided a process P8 for the preparation of a compound of formula (I) wherein Y represents a NR^{d} group wherein R^{d} represents a formyloxy group, a formylamino group, substituted or non-substituted C₁-C₈-alkyl amino, substituted or non-substituted C₁-C₈-cycloalkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₂-C₈-halogenoalkenyloxy having 1 to 5 halogen atoms, substituted or non-substituted C₃-C₈-halogenoalkynyloxy having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkoxycarbonylamino, substituted or non-substituted C₁-C₈-halogenoalkoxycarbonylamino having 1 to 5 halogen atoms, substituted or non-substituted C₁-C₈-alkoxycarbonyloxy, substituted or non-substituted C₁-C₈-halogenoalkoxycarbonyloxy having 1 to 5 halogen atoms; L² represents an oxygen atom, a sulphur atom or a NR⁹ group; Q² represents a hydrogen atom; and Q¹, p, R^{a}, R^{b}, R^{g}, L¹, as herein-defined,

and that comprises reacting a different compound of formula (I) wherein Y represents a NR^{d} group wherein R^{d} represents an amino group, a hydroxy group substituted or non-substituted C₁-C₈-alkylamino, substituted or non-substituted C₁-C₈-cycloalkylamino, L² represents an oxygen atom, a sulphur atom or a NR^{g} group; Q² represents a hydrogen atom; Q¹, p, R^{a}, R^{b}, R^{c}, R^{g}, L¹, being as herein-defined;
with a compound of formula Q²W wherein W represents a leaving group such as a halogen atom, a C₁-C₆ alkylsulfonate, a C₁-C₆ haloalkylsulfonate and Q² represents a formyl group, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₂-C₈-halogenoalkenyl having 1 to 5 halogen atoms, substituted or non-substituted C₃-C₈-halogenoalkynyl having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted C₁-C₈-alkoxycarbonyl, substituted or non-substituted C₁-C₈-halogenoalkoxycarbonyl having 1 to 5 halogen atoms; optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine.

According to a further aspect of the present invention, there is provided a process P9 for the preparation of a compound of formula (I) wherein Y represents an oxygen atom, L² represents an oxygen atom, a NR^{g} group; Q¹, p, R^{a}, R^{b}, R^{c}, R^{g}, L¹, Q², being as herein defined; and that comprises reacting a different compound of formula (I) wherein Y represents an oxygen atom, L² represents an oxygen atom and Q² represents a hydrogen atom, a formyl group, a substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, a substituted or non-substituted or a 4-, 5-, 6- or 7-membered heterocycle comprising up to 4 heteroatoms selected in the list consisting of N, O, S; Q¹, p, R^{a}, R^{b}, R^{c}, L¹, being as herein defined;

with a compound of formula Q²L²H wherein Q² being as herein defined and L² represents an oxygen atom, a NR^{g} group,
optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; in one or a different-pot conditions.

According to the present invention, the compounds of formula (I) useful as starting material within the processes P2 to P9 can be prepared according to process P1 according to the invention.

According to a further aspect of the present invention, there is provided a process P10 for the preparation of a compound of formula (I) wherein Y represents a sulphur atom, L² represents an oxygen atom, a NR^{g} group and Q¹, p, R^{a}, R^{b}, R^{c}, R^{g}, L¹, Q² being as herein defined;

and that comprises reacting a different compound of formula (I) wherein Y represents an oxygen atom, L² represents an oxygen atom, a NR^{g} group and Q¹, p, R^{a}, R^{b}, R^{c}, R^{g}, L¹, Q² being as herein defined;
with a thiocarbonylation agent such as 2,4-bis(4-methoxyphenyl)-1,3,2,4-dithiadiphosphetane 2,4-disulfide, phosphorus pentasulfide, sulphur.

According to a further aspect of the present invention, there is provided a process P11 for the preparation of a compound of formula (I) wherein Y represents a NR^{d} group or an oxygen atom, L² represents a NR^{g} group and R^{g} represents a hydrogen atom, a nitro group, a cyano group, a hydroxy group, an amino group, a formyloxy group, a formylamino group, substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, or a 4-, 5-, 6- or 7-membered heterocycle comprising up to 4 heteroatoms selected in the list consisting of N, O, S; Q¹, p, R^{a}, R^{b}, R^{c}, R^{d}, L¹, Q² being as herein defined;

and that comprises reacting a different compound of formula (I) wherein Y represents a NR^{d} group or an oxygen atom, L² represents an oxygen atom, a sulphur atom; Q² represents a formyl group, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₂-C₈-halogenoalkenyl having 1 to 5 halogen atoms, substituted or non-substituted C₃-C₈-halogenoalkynyl having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted C₁-C₈-alkoxycarbonyl, substituted or non-substituted C₁-C₈-halogenoalkoxycarbonyl having 1 to 5 halogen atoms and Q¹, p, R^{a}, R^{b}, R^{c}, R^{d}, L¹ being as herein defined; with a compound of formula R^{g}NH wherein R^{g} being as herein defined;
optionally in the presence of a base such as an inorganic or an organic base; notably an alkaline earth metal or an alkali metal hydride, hydroxide, amide, alcoholate, acetate, carbonate or hydrogen carbonate, such as sodium hydride, sodium amide, lithium diisopropylamide, sodium methanolate, sodium ethanolate, potassium tert-butanolate, sodium acetate, potassium acetate, calcium acetate, sodium hydroxide, potassium hydroxide, sodium carbonate, potassium carbonate, potassium bicarbonate, sodium bicarbonate, cesium carbonate or ammonium carbonate; and also tertiary amines, such as trimethylamine, triethylamine (TEA), tributylamine, N,N-dimethylaniline, N,N-dimethyl-benzylamine, N,N-diisopropyl-ethylamine (DIPEA), pyridine, N-methylpiperidine, N-methylmorpholine, N,N-dimethylaminopyridine, diazabicyclooctane (DABCO), diazabicyclononene (DBN) or diazabicycloundecene (DBU), optionally in the presence of an acid such as a Lewis acid; notably metal or metalloid halides such as aluminium trichloride, zinc dichloride, magnesium bromide, boron tribromide; or such as a Brönstedt acid; notably a mineral acid such as sulphuric acid, chlorhydric acid, ammonium chloride, phosphoric acid, or an organic acid, such as acetic acid, para-toluenesulphonic acid, optionally in the presence of a condensing agent such as acid halide former, notably phosgene, phosphorous tribromide, phosphorous trichloride, phosphorous pentachloride, phosphorous trichloride oxide or thionyl chloride; such as an anhydride former, notably ethyl chloroformate, methyl chloroformate, isopropyl chloroformate, isobutyl chloroformate, 2,2-dimethylpropionyl chloride or methanesulfonyl chloride; notably carbodiimides, such as N,N′-dicyclohexylcarbodiimide (DCC) or such as other customary condensing agents, notably phosphorous pentoxide, polyphosphoric acid, N,N′-carbonyldiimidazole, 2-ethoxy-N-ethoxycarbonyl-1,2-dihydroquinoline (EEDQ), triphenylphosphine/tetrachloromethane or bromo-tripyrrolidinophosphonium-hexafluorophosphate, optionally in the presence of a catalyst notably a transition metal catalyst, such as palladium salts or complexes for example palladium (II) chloride, palladium (II) acetate, tetrakis-(triphenylphosphine) palladium(0), bis-(triphenylphosphine) palladium dichloride (II), tris(dibenzylideneacetone) dipalladium(0), bis(dibenzylideneacetone) palladium(0), or 1,1′-bis(diphenylphosphino)ferrocene-palladium (II) chloride. As an alternative the palladium complex is directly generated in the reaction mixture by separately adding to the reaction mixture a palladium salt and a complex ligand such as a phosphine, for example triethylphosphine, tri-tert-butylphosphine, tricyclohexylphosphine, 2-(dicyclohexylphosphine)biphenyl, 2-(di-tert-butylphosphin)biphenyl, 2-(dicyclohexylphosphine)-2′-(N,N-dimethylamino)-biphenyl, triphenylphosphine, tris-(o-tolyl)phosphine, sodium 3-(diphenylphosphino)benzolsulfonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)-biphenyl, bis(diphenylphosphino)ferrocene, tris-(2,4-tert-butylphenyl)-phosphite, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldicyclohexylphosphine, (R)-(−)-1-[(S)-2-(diphenylphosphino)ferrocenyl]ethyldicyclohexylphosphine, (S)-(+)-1-[(R)-2-(diphenylphosphino)ferrocenyl]ethyldi-t-butylphosphine; in one or a different-pot conditions. According to the present invention, the compounds of formula (I) useful as starting material within the processes P10 to P11 can be prepared according to process P1 to P9 according to the invention.

Suitable solvents for carrying out process P1 to P11 according to the invention are in each case all customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulphoxides, such as dimethyl sulphoxide, or sulphones, such as sulpholane.

When carrying out process P1 to P11 according to the invention, the reaction temperatures can independently be varied within a relatively wide range. Generally, processes according to the invention are carried out at temperatures between −80° C. and 250° C.

Process P1 to P11 according to the invention is generally independently carried out under atmospheric pressure. However, in each case, it is also possible to operate under elevated or reduced pressure.

Work-up is carried out by customary methods. Generally, the reaction mixture is treated with water and the organic phase is separated off and, after drying, concentrated under reduced pressure. If appropriate, the remaining residue can be freed by customary methods, such as chromatography or recrystallization, from any impurities that may still be present.

Compounds according to the invention can be prepared according to the above described process. It will nevertheless be understood that, on the basis of his general knowledge and of available publications, the skilled worker will be able to adapt these processes according to the specifics of each of the compounds according to the invention that is desired to be synthesized.

Still in a further aspect, the present invention relates to compounds of formula (II) useful as intermediate compounds or materials for the process of preparation according to the invention. The present invention thus provides compounds of formula (II)

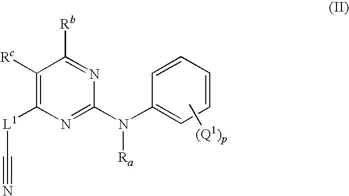

wherein Q¹, p, R^{a}, R^{b}, R^{c}, L¹ are as herein-defined.

In a further aspect, the present invention also relates to a fungicide composition comprising an effective and non-phytotoxic amount of an active compound of formula (I).

The expression “effective and non-phytotoxic amount” means an amount of composition according to the invention which is sufficient to control or destroy the fungi present or liable to appear on the crops, and which does not entail any appreciable symptom of phytotoxicity for the said crops. Such an amount can vary within a wide range depending on the fungus to be controlled, the type of crop, the climatic conditions and the compounds included in the fungicide composition according to the invention. This amount can be determined by systematic field trials, which are within the capabilities of a person skilled in the art.

Thus, according to the invention, there is provided a fungicide composition comprising, as an active ingredient, an effective amount of a compound of formula (I) as herein defined and an agriculturally acceptable support, carrier or filler.

According to the invention, the term “support” denotes a natural or synthetic, organic or inorganic compound with which the active compound of formula (I) is combined or associated to make it easier to apply, notably to the parts of the plant. This support is thus generally inert and should be agriculturally acceptable. The support may be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition according to the invention may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active compound and/or the inert support are water-insoluble and when the vector agent for the application is water. Preferably, surfactant content may be comprised from 5% to 40% by weight of the composition.

Optionally, additional components may also be included, e.g. protective colloids, adhesives, thickeners, thixotropic agents, penetration agents, stabilisers, sequestering agents. More generally, the active compounds can be combined with any solid or liquid additive, which complies with the usual formulation techniques.

In general, the composition according to the invention may contain from 0.05 to 99% by weight of active compound, preferably 10 to 70% by weight.

Compositions according to the invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. These compositions include not only compositions which are ready to be applied to the plant or seed to be treated by means of a suitable device, such as a spraying or dusting device, but also concentrated commercial compositions which must be diluted before application to the crop.

The compounds according to the invention can also be mixed with one or more insecticide, fungicide, bactericide, attractant, acaricide or pheromone active substance or other compounds with biological activity. The mixtures thus obtained have normally a broadened spectrum of activity. The mixtures with other fungicide compounds are particularly advantageous.

Examples of suitable fungicide mixing partners may be selected in the following lists:
B1) a compound capable to inhibit the nucleic acid synthesis like benalaxyl, benalaxyl-M, bupirimate, chiralaxyl, clozylacon, dimethirimol, ethirimol, furalaxyl, hymexazol, mefenoxam, metalaxyl, metalaxyl-M, ofurace, oxadixyl, oxolinic acid;
B2) a compound capable to inhibit the mitosis and cell division like benomyl, carbendazim, diethofencarb, ethaboxam, fuberidazole, pencycuron, thiabendazole thiophanate-methyl, zoxamide;
B3) a compound capable to inhibit the respiration for example as CI-respiration inhibitor like diflumetorim;
   as CII-respiration inhibitor like boscalid, carboxin, fenfuram, flutolanil, furametpyr, furmecyclox, mepronil, oxycarboxin, penthiopyrad, thifluzamide;
   as CIII-respiration inhibitor like amisulbrom, azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin;
B4) a compound capable of to act as an uncoupler like dinocap, fluazinam, meptyldinocap;
B5) a compound capable to inhibit ATP production like fentin acetate, fentin chloride, fentin hydroxide, silthiofam;
B6) a compound capable to inhibit AA and protein biosynthesis like andoprim, blasticidin-S, cyprodinil, kasugamycin, kasugamycin hydrochloride hydrate, mepanipyrim, pyrimethanil;
B7) a compound capable to inhibit the signal transduction like fenpiclonil, fludioxonil, quinoxyfen;
B8) a compound capable to inhibit lipid and membrane synthesis like chlozolinate, etridiazole, iprodione, procymidone, vinclozolin, pyrazophos, edifenphos, iprobenfos (IBP), isoprothiolane, tolclofos-methyl, biphenyl, iodocarb, propamocarb, propamocarb-hydrochloride;
B9) a compound capable to inhibit ergosterol biosynthesis like fenhexamid, azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, paclobutrazol, penconazole, propiconazole, prothioconazole, simeconazole, tebuconazole, tetraconazole, triadimefon, triadimenol, triticonazole, uniconazole, voriconazole, imazalil, imazalil sulfate, oxpoconazole, fenarimol, flurprimidol, nuarimol, pyrifenox, triforine, pefurazoate, prochloraz, triflumizole, viniconazole, aldimorph, dodemorph, dodemorph acetate, fenpropimorph, tridemorph, fenpropidin, spiroxamine, naftifine, pyributicarb, terbinafine;
B10) a compound capable to inhibit cell wall synthesis like benthiavalicarb, bialaphos, dimethomorph, flumorph, iprovalicarb, mandipropamid, polyoxins, polyoxorim, validamycin A;
B11) a compound capable to inhibit melanine biosynthesis like carpropamid, diclocymet, fenoxanil, phtalide, pyroquilon, tricyclazole;
B12) a compound capable to induce a host defence like acibenzolar-S-methyl, probenazole, tiadinil;
B13) a compound capable to have a multisite action like captafol, captan, chlorothalonil, copper preparations such as copper hydroxide, copper naphthenate, copper oxychloride, copper sulphate, copper oxide, oxine-copper and Bordeaux mixture, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, propineb, sulphur and sulphur preparations including calcium polysulphide, thiram, tolylfluanid, zineb, ziram;
B14) a compound selected in the following list: benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methylsulphate, diphenylamine, ferimzone, flumetover, flusulfamide, fosetyl-aluminium, fosetyl-calcium, fosetyl-sodium, fluopicolide, fluoroimide, hexachlorobenzene, 8-hydroxyquinoline sulfate, irumamycin, methasulphocarb, metrafenone, methyl isothiocyanate, mildiomycin, natamycin, nickel dimethyldithiocarbamate, nitrothal-isopropyl,octhilinone, oxamocarb, oxyfenthiin, pentachlorophenol and salts, 2-phenylphenol and salts, phosphorous acid and its salts, piperalin, propanosine-sodium, proquinazid, pyrroInitrine, quintozene, tecloftalam, tecnazene, triazoxide, trichlamide, zarilamid and 2,3,5,6-tetrachloro-4-(methylsulfonyl)-pyridine, N-(4-Chloro-2-nitrophenyl)-N-ethyl-4-methyl-benzenesulfonamide, 2-amino-4-methyl-N-phenyl-5-thiazolecarboxamide, 2-chloro-N-(2,3-dihydro-1,1,3-trimethyl-1H-inden-4-yl)-3-pyridincarboxamide, 3-[5-(4-chlorophenyl)-2,3-dimethylisoxazolidin-3-yl]pyridine, cis-1-(4-chlorophenyl)-2-(1H-1,2,4-triazole-1-yl)-cycloheptanol, methyl 1-(2,3-dihydro-2,2-dimethyl-1H-inden-1-yl)-1H-imidazole-5-carboxylate, 3,4,5-trichloro-2,6-pyridinedicarbonitrile, Methyl 2-[[[cyclopropyl[(4-methoxyphenyl)imino]methyl]thio]methyl]-.alpha.-(methoxymethylene)-benzeneacetate, 4-Chloro-alpha-propynyloxy-N-[2-[3-methoxy-4-(2-propynyloxy)phenyl]ethyl]-benzeneacetamide, (2S)—N-[2-[4-[[3-(4-chlorophenyl)-2-propynyl]oxy]-3-methoxyphenyl]ethyl]-3-methyl-2-[(methylsulfonyl)amino]-butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidin-7-amine, 5-chloro-N-[(1R)-1,2-dimethylpropyl]-6-(2,4,6-trifluorophenyl)[1,2,4]triazolo[1,5-a]pyrimidin-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propyl-benzopyranon-4-one, N-{(Z)-[(cyclopropylmethoxy)imino][6-(difluoromethoxy)-2,3-difluorophenyl]methyl}-2-phenylacetamide, N-(3-ethyl-3,5,5-trimethyl-cyclohexyl)-3-formylamino-2-hydroxy-benzamide, 2-[[[[1-[3(1-Fluoro-2-phenylethyl)oxy]phenyl]ethylidene]amino]oxy]methyl]-alpha-(methoxyimino)-N-methyl-alphaE-benzeneacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, 2-(2-{[6-(3-chloro-2-methylphenoxy)-5-fluoropyrimidin-4-yl]oxy}phenyl)-2-(methoxyimino)-N-methylacetamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-{2-[1,1′-bi(cyclopropyl)-2-yl]phenyl}-3-(difluoromethyl)-, 1-methyl-1H-pyrazole-4-carboxamide, N-ethyl-N-methyl-N′-{2-methyl-5-(trifluoromethyl)-4-[3-(trimethylsilyl)propoxy]phenyl}imidoformamide, pyribencarb, valiphenal.

The composition according to the invention comprising a mixture of a compound of formula (I) with a bactericide compound may also be particularly advantageous. Examples of suitable bactericide mixing partners may be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

The compounds of formula (I) and the fungicide composition according to the invention can be used to curatively or preventively control the phytopathogenic fungi of plants or crops. Thus, according to a further aspect of the invention, there is provided a method for curatively or preventively controlling the phytopathogenic fungi of plants or crops characterised in that a compound of formula (I) or a fungicide composition according to the invention is applied to the seed, the plant or to the fruit of the plant or to the soil wherein the plant is growing or wherein it is desired to grow.

The method of treatment according to the invention may also be useful to treat propagation material such as tubers or rhizomes, but also seeds, seedlings or seedlings pricking out and plants or plants pricking out. This method of treatment can also be useful to treat roots. The method of treatment according to the invention can also be useful to treat the over ground parts of the plant such as trunks, stems or stalks, leaves, flowers and fruit of the concerned plant.

Among the plants that can be protected by the method according to the invention, mention may be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for instance banana trees and plantins), Rubiaceae sp., Theaceae sp., Sterculiceae sp., Rutaceae sp. (for instance lemons, oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

Among the diseases of plants or crops that can be controlled by the method according to the invention, mention may be made of:
Powdery mildew diseases such as:
Blumeria diseases, caused for example by Blumeria graminis;
Podosphaera diseases, caused for example by Podosphaera leucotricha;
Sphaerotheca diseases, caused for example by Sphaerotheca fuliginea;
Uncinula diseases, caused for example by Uncinula necator;
Rust diseases such as:
Gymnosporangium diseases, caused for example by Gymnosporangium sabinae;
Hemileia diseases, caused for example by Hemileia vastatrix;
Phakopsora diseases, caused for example by Phakopsora pachyrhizi or Phakopsora meibomiae;
Puccinia diseases, caused for example by Puccinia recondita;
Uromyces diseases, caused for example by Uromyces appendiculatus;
Oomycete diseases such as:
Bremia diseases, caused for example by Bremia lactucae;
Peronospora diseases, caused for example by Peronospora pisi or P. brassicae;
Phytophthora diseases, caused for example by Phytophthora infestans;
Plasmopara diseases, caused for example by Plasmopara viticola;
Pseudoperonospora diseases, caused for example by Pseudoperonospora humuli or Pseudoperonospora cubensis;
Pythium diseases, caused for example by Pythium ultimum;
Leafspot, leaf blotch and leaf blight diseases such as:
Alternaria diseases, caused for example by Alternaria solani;
Cercospora diseases, caused for example by Cercospora beticola;
Cladiosporum diseases, caused for example by Cladiosporium cucumerinum;
Cochliobolus diseases, caused for example by Cochliobolus sativus;
Colletotrichum diseases, caused for example by Colletotrichum lindemuthanium;
Cycloconium diseases, caused for example by Cycloconium oleaginum;
Diaporthe diseases, caused for example by Diaporthe citri;
Elsinoe diseases, caused for example by Elsinoe fawcettii;
Gloeosporium diseases, caused for example by Gloeosporium laeticolor;
Glomerella diseases, caused for example by Glomerella cingulata;
Guignardia diseases, caused for example by Guignardia bidwelli;
Leptosphaeria diseases, caused for example by Leptosphaeria maculans;
Leptosphaeria nodorum;
Magnaporthe diseases, caused for example by Magnaporthe grisea;
Mycosphaerella diseases, caused for example by Mycosphaerella graminicola;
Mycosphaerella arachidicola; Mycosphaerella fijiensis;
Phaeosphaeria diseases, caused for example by Phaeosphaeria nodorum;
Pyrenophora diseases, caused for example by Pyrenophora teres;
Ramularia diseases, caused for example by Ramularia collo-cygni;
Rhynchosporium diseases, caused for example by Rhynchosporium secalis;
Septoria diseases, caused for example by Septoria apii or Septoria lycopercisi;
Typhula diseases, caused for example by Typhula incamata;
Venturia diseases, caused for example by Venturia inaequalis;
Root and stem diseases such as:
Corticium diseases, caused for example by Corticium graminearum;
Fusarium diseases, caused for example by Fusarium oxysporum;
Gaeumannomyces diseases, caused for example by Gaeumannomyces graminis;
Rhizoctonia diseases, caused for example by Rhizoctonia solani;
Tapesia diseases, caused for example by Tapesia acuformis;
Thielaviopsis diseases, caused for example by Thielaviopsis basicola;
Ear and panicle diseases such as:
Alternaria diseases, caused for example by Alternaria spp.;
Aspergillus diseases, caused for example by Aspergillus flavus;
Cladosporium diseases, caused for example by Cladosporium spp.;
Claviceps diseases, caused for example by Claviceps purpurea;
Fusarium diseases, caused for example by Fusarium culmorum;
Gibberella diseases, caused for example by Gibberella zeae;
Monographella diseases, caused for example by Monographella nivalis;
Smut and bunt diseases such as:
Sphacelotheca diseases, caused for example by Sphacelotheca reiliana;
Tilletia diseases, caused for example by Tilletia caries;
Urocystis diseases, caused for example by Urocystis occulta;
Ustilago diseases, caused for example by Ustilago nuda;
Fruit rot and mould diseases such as:
Aspergillus diseases, caused for example by Aspergillus flavus;
Botrytis diseases, caused for example by Botrytis cinerea;
Penicillium diseases, caused for example by Penicillium expansum;
Sclerotinia diseases, caused for example by Sclerotinia sclerotiorum;
Verticilium diseases, caused for example by Verticilium alboatrum;
Seed and soilborne decay, mould, wilt, rot and damping-off diseases such as:
Fusarium diseases, caused for example by Fusarium culmorum;
Phytophthora diseases, caused for example by Phytophthora cactorum;
Pythium diseases, caused for example by Pythium ultimum;
Rhizoctonia diseases, caused for example by Rhizoctonia solani;
Sclerotium diseases, caused for example by Sclerotium rolfsii;
Microdochium diseases, caused for example by Microdochium nivale;
Canker, broom and dieback diseases such as:
Nectria diseases, caused for example by Nectria galligena;
Blight diseases such as:
Monilinia diseases, caused for example by Monilinia laxa;
Leaf blister or leaf curl diseases such as:
Taphrina diseases, caused for example by Taphrina deformans;
Decline diseases of wooden plants such as:
Esca diseases, caused for example by Phaemoniella clamydospora;
Eutypa dyeback, caused for example by Eutypa lata;
Dutch elm disease, caused for example by Ceratocystsc ulmi;
Diseases of flowers and Seeds such as:
Botrytis diseases, caused for example by Botrytis cinerea;
Diseases of tubers such as:
Rhizoctonia diseases, caused for example by Rhizoctonia solani.

The fungicide composition according to the invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds according to the invention, or a composition according to the invention; this includes for example direct application, spraying, dipping, injection or any other suitable means.

The dose of active compound usually applied in the method of treatment according to the invention is generally and advantageously from 10 to 800 g/ha, preferably from 50 to 300 g/ha for applications in foliar treatment. The dose of active substance applied is generally and advantageously from 2 to 200 g per 100 kg of seed, preferably from 3 to 150 g per 100 kg of seed in the case of seed treatment.

It is clearly understood that the doses indicated herein are given as illustrative examples of the method according to the invention. A person skilled in the art will know how to adapt the application doses, notably according to the nature of the plant or crop to be treated.

The fungicide composition according to the invention may also be used in the treatment of genetically modified organisms with the compounds according to the invention or the agrochemical compositions according to the invention. Genetically modified plants are plants into genome of which a heterologous gene encoding a protein of interest has been stably integrated. The expression “heterologous gene encoding a protein of interest” essentially means genes which give the transformed plant new agronomic properties, or genes for improving the agronomic quality of the modified plant.

The compounds or mixtures according to the invention may also be used for the preparation of composition useful to curatively or preventively treat human or animal fungal diseases such as, for example, mycoses, dermatoses, trichophyton diseases and candidiases or diseases caused by Aspergillus spp., for example Aspergillus fumigatus.

The various aspects of the invention will now be illustrated with reference to the following tables A and B of compound examples and the following preparation or efficacy examples.

The following tables A, B and C illustrate in a non-limiting manner examples of compounds according to the invention.

In the following tables, M+H (or M−H) means the molecular ion peak, plus or minus 1 a.m.u. (atomic mass unit) respectively, as observed in mass spectroscopy and M (Apcl+) means the molecular ion peak as it was found via positive atmospheric pressure chemical ionisation in mass spectroscopy.

TABLE A

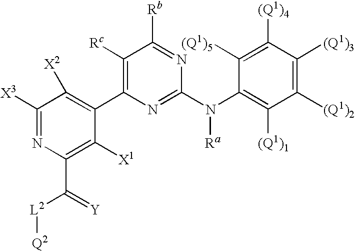

| Exam- | | | | | | | | | | | | | | | | |
|---|---|---|---|---|---|---|---|---|---|---|---|---|---|---|---|---|
| ple | | | | | | | | | | | | | | | Measured | |
| Num- | | | | | | | | | | | | | | | MW or | |
| ber | L² | Q² | Y | (Q¹)¹ | (Q¹)² | (Q¹)³ | (Q¹)⁴ | (Q¹)⁵ | X¹ | X² | X³ | R^{a} | R^{b} | R^{c} | ¹H NMR | Log p |
| A1 | NH | 3-(2-Oxo- | O | H | Cl | H | H | H | H | H | H | H | H | H | 479 | 2.98 |
| | | azepan-1- | | | | | | | | | | | | | | |
| | | yl)propyl | | | | | | | | | | | | | | |
| A2 | O | Ethyl | O | H | Cl | H | H | H | H | H | H | H | H | H | 455 | 2.75 |
| A3 | O | 2-Methoxy- | O | H | Cl | H | H | H | H | H | H | H | H | H | 385 | 2.82 |
| | | ethyl | | | | | | | | | | | | | | |
| A4 | NH | 3-(2-Oxo- | O | H | CH₃O | H | H | H | H | H | H | H | H | H | 475 | 2.5 |
| | | azepan-1- | | | | | | | | | | | | | | |
| | | yl)propyl | | | | | | | | | | | | | | |
| A5 | O | H | O | H | CH₃CO | H | H | H | H | H | H | H | H | H | 335 | 1.36 |
| A6 | O | H | O | H | H | F | H | H | H | H | H | H | H | H | 311 | 1.58 |
| A7 | O | H | O | H | H | CH₃CONH | H | H | H | H | H | H | H | H | 350 | 0.83 |
| A8 | O | H | O | H | H | CH₃CO | H | H | H | H | H | H | H | H | 335 | 1.31 |
| A9 | O | H | O | H | CH₃ | F | H | H | H | H | H | H | H | H | 325 | 1.84 |
| A10 | O | H | O | H | Nitro | H | H | H | H | H | H | H | H | H | 338 | 1.66 |
| A11 | O | 2-Methoxy- | O | H | H | CH₃CO | H | H | H | H | H | H | H | H | 393 | 1.97 |
| | | ethyl | | | | | | | | | | | | | | |
| A12 | O | 2-Methoxy- | O | H | CH₃ | F | H | H | H | H | H | H | H | H | 383 | 2.64 |
| | | ethyl | | | | | | | | | | | | | | |
| A13 | O | 2-Methoxy- | O | H | CH₃CONH | H | H | H | H | H | H | H | H | H | 408 | 1.61 |
| | | ethyl | | | | | | | | | | | | | | |
| A14 | O | 2-Methoxy- | O | H | H | CH₃SO₂ | H | H | H | H | H | H | H | H | 429 | 1.73 |
| | | ethyl | | | | | | | | | | | | | | |
| A15 | O | 2-Methoxy- | O | H | CH₃O | CH₃O | Meth- | H | H | H | H | H | H | H | 442 | 1.99 |
| | | ethyl | | | | | oxy | | | | | | | | | |
| A16 | O | 2-Methoxy- | O | H | CH₃O | H | H | H | H | H | H | H | H | H | 381 | 2.27 |
| | | ethyl | | | | | | | | | | | | | | |
| A17 | O | Li | O | H | Cl | H | H | H | H | H | H | H | H | H | 8.05 | |
| | | | | | | | | | | | | | | | (s, 1H); | |
| | | | | | | | | | | | | | | | 7.95 | |
| | | | | | | | | | | | | | | | (d, 1H); | |
| | | | | | | | | | | | | | | | 7.80 | |
| | | | | | | | | | | | | | | | (dd, 1H); | |
| | | | | | | | | | | | | | | | 7.55 | |
| | | | | | | | | | | | | | | | (d, 1H); | |
| | | | | | | | | | | | | | | | 7.35 | |
| | | | | | | | | | | | | | | | (t, 1H); | |
| | | | | | | | | | | | | | | | 7.05 | |
| A18 | O | H | O | H | H | 2-Oxo- | H | H | H | H | H | H | H | H | 376 | 1.17 |
| | | | | | | pyrrolidin- | | | | | | | | | | |
| | | | | | | 1-yl | | | | | | | | | | |
| A19 | NH | 3-Methyl- | O | H | Cl | H | H | H | H | H | H | H | H | H | 396 | 4.7 |
| | | butyl | | | | | | | | | | | | | | |
| A20 | O | 2-Methoxy- | O | H | H | 2-Oxo- | H | H | H | H | H | H | H | H | 434 | 1.79 |
| | | ethyl | | | | pyrrolidin- | | | | | | | | | | |
| | | | | | | 1-yl | | | | | | | | | | |
| A21 | O | 2-Methoxy- | O | H | Nitro | H | H | H | H | H | H | H | H | H | 396 | 2.4 |
| | | ethyl | | | | | | | | | | | | | | |
| A22 | NH | 1-Methyl- | O | H | Cl | H | H | H | H | H | H | H | H | H | 396 | 4.51 |
| | | butyl | | | | | | | | | | | | | | |
| A23 | NH | 2-Methoxy- | O | H | Cl | H | H | H | H | H | H | H | H | H | 384 | 3.03 |
| | | ethyl | | | | | | | | | | | | | | |
| A24 | O | 2-Methoxy- | O | H | Cl | H | H | H | H | H | H | H | H | H | 399 | 3.15 |
| | | 1-methyl- | | | | | | | | | | | | | | |
| | | ethyl | | | | | | | | | | | | | | |
| A25 | O | 1-Methyl- | O | H | Cl | H | H | H | H | H | H | H | H | H | 397 | 4.54 |
| | | butyl | | | | | | | | | | | | | | |
| A26 | O | Pentyl | O | H | Cl | H | H | H | H | H | H | H | H | H | 397 | 4.62 |
| A27 | O | Methyl | O | H | Cl | H | H | H | H | H | H | H | H | H | 341 | 2.92 |
| A28 | O | Butyl | O | H | Cl | H | H | H | H | H | H | H | H | H | 383 | 4.16 |
| A29 | NH | 2-Methoxy- | O | H | Cl | H | H | H | H | H | H | H | H | H | 398 | 3.6 |
| | | 1-methyl- | | | | | | | | | | | | | | |
| | | ethyl | | | | | | | | | | | | | | |
| A30 | NH | 4-(Dimethyl- | O | H | Cl | H | H | H | H | H | H | H | H | H | 425 | 1.84 |
| | | amino)- | | | | | | | | | | | | | | |
| | | butyl | | | | | | | | | | | | | | |
| A31 | NH | 2-(Dimethyl- | O | H | Cl | H | H | H | H | H | H | H | H | H | 397 | 1.74 |
| | | amino)- | | | | | | | | | | | | | | |
| | | ethyl | | | | | | | | | | | | | | |
| A32 | NH | 3-(Dimethyl- | O | H | Cl | H | H | H | H | H | H | H | H | H | 411 | 1.77 |
| | | amino)- | | | | | | | | | | | | | | |
| | | propyl | | | | | | | | | | | | | | |
| A33 | NH | Tetrahydro- | O | H | Cl | H | H | H | H | H | H | H | H | H | 410 | 3.46 |
| | | furan-2- | | | | | | | | | | | | | | |
| | | ylmethyl | | | | | | | | | | | | | | |
| A34 | NH | Cyclohexyl | O | H | Cl | H | H | H | H | H | H | H | H | H | 408 | 4.9 |
| A35 | NH | 1,2- | O | H | Cl | H | H | H | H | H | H | H | H | H | 396 | 4.67 |
| | | Dimethyl- | | | | | | | | | | | | | | |
| | | propyl | | | | | | | | | | | | | | |
| A36 | NH | Cyclopropyl | O | H | Cl | H | H | H | H | H | H | H | H | H | 366 | 3.37 |
| A37 | NH | 4-(2-Oxo- | O | H | Cl | H | H | H | H | H | H | H | H | H | 465 | 2.84 |
| | | pyrrolidin- | | | | | | | | | | | | | | |
| | | 1-yl)butyl | | | | | | | | | | | | | | |
| A38 | NH | 1-(Dimethyl- | O | H | Cl | H | H | H | H | H | H | H | H | H | 411 | 1.81 |
| | | amino)- | | | | | | | | | | | | | | |
| | | propan-2-yl | | | | | | | | | | | | | | |
| A39 | NH | 3-Methoxy- | O | H | Cl | H | H | H | H | H | H | H | H | H | 398 | 3.31 |
| | | propyl | | | | | | | | | | | | | | |
| A40 | NH | Cyclopentyl | O | H | Cl | H | H | H | H | H | H | H | H | H | 394 | 4.48 |
| A41 | NH | 2(S)-1- | O | H | Cl | H | H | H | H | H | H | H | H | H | 398 | 3.62 |
| | | Methoxy- | | | | | | | | | | | | | | |
| | | propan-2- | | | | | | | | | | | | | | |
| | | yl | | | | | | | | | | | | | | |
| A42 | NH | 3-(2-Oxo- | O | H | Cl | H | H | H | H | H | H | H | H | H | 451 | 2.75 |
| | | pyrrolidin- | | | | | | | | | | | | | | |
| | | 1-yl)propyl | | | | | | | | | | | | | | |
| A43 | NH | Pentyl | O | H | Cl | H | H | H | H | H | H | H | H | H | 396 | 4.82 |
| A44 | NH | 1-Ethyl- | O | H | Cl | H | H | H | H | H | H | H | H | H | 396 | 4.72 |
| | | propyl | | | | | | | | | | | | | | |
| A45 | NH | 1-Methoxy- | O | H | Cl | H | H | H | H | H | H | H | H | H | 412 | 4.02 |
| | | butan-2- | | | | | | | | | | | | | | |
| | | yl | | | | | | | | | | | | | | |
| A46 | NH | sec-Butyl | O | H | Cl | H | H | H | H | H | H | H | H | H | 382 | 4.3 |
| A47 | NH | Cyclobutyl | O | H | Cl | H | H | H | H | H | H | H | H | H | 380 | 4.1 |
| A48 | NH | tert-Butyl | O | H | Cl | H | H | H | H | H | H | H | H | H | 382 | 4.67 |
| A49 | NH | Cyclopropyl- | O | H | Cl | H | H | H | H | H | H | H | H | H | 380 | 3.97 |
| | | methyl | | | | | | | | | | | | | | |
| A50 | NH | 2- | O | H | Cl | H | H | H | H | H | H | H | H | H | 439 | 1.75 |
| | | Morpholin- | | | | | | | | | | | | | | |
| | | 4-ylethyl | | | | | | | | | | | | | | |
| A51 | NH | 2-Methoxy- | O | H | Cl | H | H | H | H | H | H | H | H | H | 412 | 3.55 |
| | | 1-methyl- | | | | | | | | | | | | | | |
| | | 2-oxoethyl | | | | | | | | | | | | | | |
| A52 | NH | 1-(Diethyl- | O | H | Cl | H | H | H | H | H | H | H | H | H | 439 | 1.96 |
| | | amino)- | | | | | | | | | | | | | | |
| | | propan-2-yl | | | | | | | | | | | | | | |
| A53 | NH | 1,3- | O | H | Cl | H | H | H | H | H | H | H | H | H | 428 | 3.55 |
| | | Dimethoxy- | | | | | | | | | | | | | | |
| | | propan- | | | | | | | | | | | | | | |
| | | 2-yl | | | | | | | | | | | | | | |
| A54 | NH | 1-Cyano- | O | H | Cl | H | H | H | H | H | H | H | H | H | 407 | 3.53 |
| | | butan-2-yl | | | | | | | | | | | | | | |
| A55 | NH | Ethyl | O | H | Cl | H | H | H | H | H | H | H | H | H | 354 | 3.35 |
| A56 | NH | Propyl | O | H | Cl | H | H | H | H | H | H | H | H | H | 368 | 3.8 |
| A57 | NH | Isopropyl | O | H | Cl | H | H | H | H | H | H | H | H | H | 368 | 3.83 |
| A58 | NH | Butyl | O | H | Cl | H | H | H | H | H | H | H | H | H | 382 | 4.31 |
| A59 | NH | Methyl | O | H | Cl | H | H | H | H | H | H | H | H | H | 340 | 2.94 |
| A60 | NH | 1,1- | O | H | Cl | H | H | H | H | H | H | H | H | H | 444 | 2.8 |
| | | Dioxido- | | | | | | | | | | | | | | |
| | | tetrahydro- | | | | | | | | | | | | | | |
| | | thiophen- | | | | | | | | | | | | | | |
| | | 3-yl | | | | | | | | | | | | | | |
| A61 | NH | 4-Ethoxy- | O | H | Cl | H | H | H | H | H | H | H | H | H | 452 | 4.54 |
| | | cyclohexyl | | | | | | | | | | | | | | |
| | | (trans- | | | | | | | | | | | | | | |
| | | dia- | | | | | | | | | | | | | | |
| | | stereomer) | | | | | | | | | | | | | | |
| A62 | NH | 4-Ethoxy- | O | H | Cl | H | H | H | H | H | H | H | H | H | 452 | 4.26 |
| | | cyclohexyl | | | | | | | | | | | | | | |
| | | (cis-dia- | | | | | | | | | | | | | | |
| | | stereomer) | | | | | | | | | | | | | | |
| A65 | NH | 4-Hydroxy- | O | H | Cl | H | H | H | H | H | H | H | H | H | 398 | 2.64 |
| | | butyl | | | | | | | | | | | | | | |
| A66 | NH | 3-(Formyl- | O | H | Cl | H | H | H | H | H | H | H | H | H | 411 | 2.39 |
| | | amino)- | | | | | | | | | | | | | | |
| | | propyl | | | | | | | | | | | | | | |
| A67 | O | Ethyl | O | H | H | H | H | H | H | H | H | H | H | H | 398 | 2.46 |
| A68 | O | Isopropyl | O | H | Cl | H | H | H | H | H | H | H | H | H | 411 | 3.64 |
| A69 | O | 3-Methoxy- | O | H | Cl | H | H | H | H | H | H | H | H | H | 321 | 3.23 |
| | | propyl | | | | | | | | | | | | | | |
| A70 | O | But-3-yn- | O | H | Cl | H | H | H | H | H | H | H | H | H | 369 | 3.35 |
| | | 1-yl | | | | | | | | | | | | | | |
| A71 | O | 3-Methylbut- | O | H | Cl | H | H | H | H | H | H | H | H | H | 399 | 4.24 |
| | | 2-en- | | | | | | | | | | | | | | |
| | | 1-yl | | | | | | | | | | | | | | |
| A72 | O | 3-Methylbut- | O | H | Cl | H | H | H | H | H | H | H | H | H | 379 | 4.16 |
| | | 3-en- | | | | | | | | | | | | | | |
| | | 1-yl | | | | | | | | | | | | | | |
| A73 | O | 1-Methyl- | O | H | Cl | H | H | H | H | H | H | H | H | H | 395 | 4.94 |
| | | pentyl | | | | | | | | | | | | | | |
| A74 | CH₃NH | 1,3- | O | H | Cl | H | H | H | H | H | H | H | H | H | 395 | 2.94 |
| | | Dimethoxy- | | | | | | | | | | | | | | |
| | | propan- | | | | | | | | | | | | | | |
| | | 2-yl | | | | | | | | | | | | | | |
| A75 | CH₃NH | Ethyl | O | H | Cl | H | H | H | H | H | H | H | H | H | 411 | 2.84 |
| A76 | CH₃NH | Methyl | O | H | Cl | H | H | H | H | H | H | H | H | H | 442 | 2.5 |
| A77 | CH₃CH₂NH | Ethyl | O | H | Cl | H | H | H | H | H | H | H | H | H | 368 | 3.19 |
| A78 | 2,5-Dimethyl-2,5-dihydro-1H- | | O | H | Cl | H | H | H | H | H | H | H | H | H | 406 | 3.75 |
| | pyrrol-1-yl | | | | | | | | | | | | | | | |
| A79 | Azetidin-1-yl | | O | H | Cl | H | H | H | H | H | H | H | H | H | 366 | 3.13 |
| A80 | 3,3-Dimethyl-piperidin-1-yl | | O | H | Cl | H | H | H | H | H | H | H | H | H | 422 | 3.96 |
| A81 | Thiomorpholin-4-yl | | O | H | Cl | H | H | H | H | H | H | H | H | H | 412 | 3.06 |
| A82 | 3,6-Dihydropyridin-1(2H)-yl | | O | H | Cl | H | H | H | H | H | H | H | H | H | 392 | 3.17 |
| A83 | 4-Hydroxypiperidin-1-yl | | O | H | Cl | H | H | H | H | H | H | H | H | H | 410 | 2.14 |
| A84 | 2,6-Dimethylmorpholin-4-yl | | O | H | Cl | H | H | H | H | H | H | H | H | H | 424 | 3.08 |
| A85 | 4-Methylpiperazin-1-yl | | O | H | Cl | H | H | H | H | H | H | H | H | H | 409 | 1.51 |
| A86 | Piperidin-1-yl | | O | H | Cl | H | H | H | H | H | H | H | H | H | 394 | 3.21 |
| A87 | Pyrrolidin-1-yl | | O | H | Cl | H | H | H | H | H | H | H | H | H | 379 | 3.06 |
| A88 | Morpholin-4-yl | | O | H | Cl | H | H | H | H | H | H | H | H | H | 396 | 2.51 |
| A89 | 2-Methylpyrrolidin-1-yl | | O | H | Cl | H | H | H | H | H | H | H | H | H | 394 | 3.37 |
| A90 | 3,5-Dimethylpiperidin-1-yl | | O | H | Cl | H | H | H | H | H | H | H | H | H | 422 | 4.06 |
| A91 | direct bond | Methyl | NOH | H | Cl | H | H | H | H | H | H | H | H | H | 340 | 3.05 |
| A92 | direct bond | Ethyl | NOH | H | Cl | H | H | H | H | H | H | H | H | H | 354 | 3.51 |
| A93 | direct bond | Methyl | NOCH₃ | H | Cl | H | H | H | H | H | H | H | H | H | 354 | 4.46 |
| A94 | direct bond | Ethyl | NOCH₃ | H | Cl | H | H | H | H | H | H | H | H | H | 368 | 5.08 |
| A95 | direct bond | Methyl | NOCH₂CH₃ | H | Cl | H | H | H | H | H | H | H | H | H | 368 | 4.92 |
| A96 | direct bond | Ethyl | NOCH₂CH₃ | H | Cl | H | H | H | H | H | H | H | H | H | 382 | 5.51 |
| A97 | direct bond | Methyl | NOallyl | H | Cl | H | H | H | H | H | H | H | H | H | 370 | 5.00 |
| A98 | direct bond | Ethyl | NOallyl | H | Cl | H | H | H | H | H | H | H | H | H | 394 | 5.54 |
| A99 | direct bond | Methyl | NNHCH₂CF₃ | H | Cl | H | H | H | H | H | H | H | H | H | 421 | 3.44 |
| A100 | direct bond | Ethyl | NNHCH₂CF₃ | H | Cl | H | H | H | H | H | H | H | H | H | 435 | 3.71 |
| A101 | direct bond | Ethyl | NNHCH₃ | H | Cl | H | H | H | H | H | H | H | H | H | 367 | 2.32 |
| A102 | direct bond | Methy | NOCH₂CF₃ | H | Cl | H | H | H | H | H | H | H | H | H | 422 | 4.94 |
| A103 | direct bond | Ethyl | NOCH₂CF₃ | H | Cl | H | H | H | H | H | H | H | H | H | 436 | 5.42 |
| A104 | direct bond | Ethyl | NNH₂ | H | Cl | H | H | H | H | H | H | H | H | H | 353 | 2.23 |
| A105 | direct bond | Methyl | 4- | H | Cl | H | H | H | H | H | H | H | H | H | 449 | 4.94 |
| | | | Chlorphenyl- | | | | | | | | | | | | | |
| | | | NHN | | | | | | | | | | | | | |
| A106 | direct bond | Methyl | NNH₂ | H | Cl | H | H | H | H | H | H | H | H | H | 337 | 2.06 |
| A107 | direct bond | Ethyl | 2-Cyanoethyl- | H | Cl | H | H | H | H | H | H | H | H | H | 406 | 2.60 |
| | | | NHN | | | | | | | | | | | | | |
| A108 | direct bond | Methyl | 2-Cyanoethyl- | H | Cl | H | H | H | H | H | H | H | H | H | 392 | 2.44 |
| | | | NHN | | | | | | | | | | | | | |
| A109 | direct bond | Ethyl | 4- | H | Cl | H | H | H | H | H | H | H | H | H | 463 | 5.34 |
| | | | Chlorphenyl- | | | | | | | | | | | | | |
| | | | NHN | | | | | | | | | | | | | |
| A110 | direct bond | Methyl | O | H | Cl | H | H | H | H | H | H | H | H | H | 325 | 3.50 |
| A111 | direct bond | Ethyl | O | H | Cl | H | H | H | H | H | H | H | H | H | 339 | 4.14 |
| A112 | CH₃NH | Ethyl | O | H | H | F | H | H | H | H | H | H | H | H | 352 | 2.42 |
| A113 | CH₃NH | Allyl | O | H | H | F | H | H | H | H | H | H | H | H | 364 | 2.66 |
| A114 | CH₃CH₂NH | Propyl | O | H | H | F | H | H | H | H | H | H | H | H | 380 | 3.08 |
| A115 | CH₃NH | Butyl | O | H | H | F | H | H | H | H | H | H | H | H | 380 | 3.11 |
| A116 | CH₃NH | Propyl | O | H | H | F | H | H | H | H | H | H | H | H | 366 | 2.73 |
| A117 | CH₃CH₂NH | Ethyl | O | H | H | F | H | H | H | H | H | H | H | H | 366 | 2.73 |
| A118 | CH₃NH | Cyclopropyl- | O | H | H | F | H | H | H | H | H | H | H | H | 378 | 2.84 |
| | | methyl | | | | | | | | | | | | | | |
| A119 | CH₃NH | Isopropyl | O | H | H | F | H | H | H | H | H | H | H | H | 366 | 2.66 |
| A120 | CH₃NH | sec-Butyl | O | H | H | F | H | H | H | H | H | H | H | H | 380 | 2.96 |
| A121 | CH₃NH | Methyl | O | H | H | F | H | H | H | H | H | H | H | H | 338 | 2.14 |
| A122 | CH₃NH | Isobutyl | O | H | H | F | H | H | H | H | H | H | H | H | 380 | 3.04 |
| A123 | CH₃CH₂CH₂NH | Cyclopropyl- | O | H | H | F | H | H | H | H | H | H | H | H | 406 | 3.53 |
| | | methyl | | | | | | | | | | | | | | |
| A124 | CH₃NH | 2-Methoxy- | O | H | H | F | H | H | H | H | H | H | H | H | 382 | 2.30 |
| | | ethyl | | | | | | | | | | | | | | |
| A125 | CH₃NH | Ethyl | O | H | CH₃ | F | H | H | H | H | H | H | H | H | 366 | 2.73 |
| A126 | CH₃NH | Allyl | O | H | CH₃ | F | H | H | H | H | H | H | H | H | 378 | 3.00 |
| A127 | CH₃CH₂NH | Propyl | O | H | CH₃ | F | H | H | H | H | H | H | H | H | 394 | 3.44 |
| A128 | CH₃NH | Butyl | O | H | CH₃ | F | H | H | H | H | H | H | H | H | 394 | 3.44 |
| A129 | CH₃NH | Propyl | O | H | CH₃ | F | H | H | H | H | H | H | H | H | 380 | 3.08 |
| A130 | CH₃CH₂NH | Ethyl | O | H | CH₃ | F | H | H | H | H | H | H | H | H | 380 | 3.06 |
| A131 | CH₃NH | Cyclopropyl- | O | H | CH₃ | F | H | H | H | H | H | H | H | H | 392 | 3.17 |
| | | methyl | | | | | | | | | | | | | | |
| A132 | CH₃NH | Isopropyl | O | H | CH₃ | F | H | H | H | H | H | H | H | H | 380 | 2.98 |
| A133 | CH₃NH | sec-Butyl | O | H | CH₃ | F | H | H | H | H | H | H | H | H | 394 | 3.31 |
| A134 | CH₃NH | Methyl | O | H | CH₃ | F | H | H | H | H | H | H | H | H | 352 | 2.42 |
| A135 | CH₃NH | Isobutyl | O | H | CH₃ | F | H | H | H | H | H | H | H | H | 394 | 3.37 |
| A136 | CH₃CH₂CH₂NH | Cyclopropyl- | O | H | CH₃ | F | H | H | H | H | H | H | H | H | 420 | 3.89 |
| | | methyl | | | | | | | | | | | | | | |
| A137 | CH₃NH | 2-Methoxy- | O | H | CH₃ | F | H | H | H | H | H | H | H | H | 396 | 2.59 |
| | | ethyl | | | | | | | | | | | | | | |
| A138 | CH₃NH | Ethyl | O | H | CH₃O | H | H | H | H | H | H | H | H | H | 364 | 2.37 |
| A139 | CH₃NH | Allyl | O | H | CH₃O | H | H | H | H | H | H | H | H | H | 376 | 2.61 |
| A140 | CH₃CH₂NH | Propyl | O | H | CH₃O | H | H | H | H | H | H | H | H | H | 392 | 3.04 |
| A141 | CH₃NH | Butyl | O | H | CH₃O | H | H | H | H | H | H | H | H | H | 392 | 3.06 |
| A142 | CH₃NH | Propyl | O | H | CH₃O | H | H | H | H | H | H | H | H | H | 378 | 2.68 |
| A143 | CH₃CH₂NH | Ethyl | O | H | CH₃O | H | H | H | H | H | H | H | H | H | 378 | 2.66 |
| A144 | CH₃NH | Cyclopropyl- | O | H | CH₃O | H | H | H | H | H | H | H | H | H | 390 | 2.78 |
| | | methyl | | | | | | | | | | | | | | |
| A145 | CH₃NH | Isopropyl | O | H | CH₃O | H | H | H | H | H | H | H | H | H | 378 | 2.61 |
| A146 | CH₃NH | sec-Butyl | O | H | CH₃O | H | H | H | H | H | H | H | H | H | 392 | 2.92 |
| A147 | CH₃NH | Methyl | O | H | CH₃O | H | H | H | H | H | H | H | H | H | 350 | 2.08 |
| A148 | CH₃NH | Isobutyl | O | H | CH₃O | H | H | H | H | H | H | H | H | H | 392 | 2.98 |
| A149 | CH₃CH₂CH₂NH | Cyclopropyl- | O | H | CH₃O | H | H | H | H | H | H | H | H | H | 418 | 3.48 |
| | | methyl | | | | | | | | | | | | | | |
| A150 | CH₃NH | 2-Methoxy- | O | H | CH₃O | F | H | H | H | H | H | H | H | H | 394 | 2.23 |
| | | ethyl | | | | | | | | | | | | | | |
| A151 | CH₃NH | Ethyl | O | H | CH₃CONH | H | H | H | H | H | H | H | H | H | 391 | 1.67 |
| A152 | CH₃NH | Allyl | O | H | CH₃CONH | H | H | H | H | H | H | H | H | H | 403 | 1.87 |
| A153 | CH₃CH₂NH | Propyl | O | H | CH₃CONH | H | H | H | H | H | H | H | H | H | 419 | 2.17 |
| A154 | CH₃NH | Butyl | O | H | CH₃CONH | H | H | H | H | H | H | H | H | H | 419 | 2.18 |
| A155 | CH₃NH | Propyl | O | H | CH₃CONH | H | H | H | H | H | H | H | H | H | 405 | 1.91 |
| A156 | CH₃CH₂NH | Ethyl | O | H | CH₃CONH | H | H | H | H | H | H | H | H | H | 405 | 1.91 |
| A157 | CH₃NH | Cyclopropyl- | O | H | CH₃CONH | H | H | H | H | H | H | H | H | H | 417 | 2.01 |
| | | methyl | | | | | | | | | | | | | | |
| A158 | CH₃NH | Isopropyl | O | H | CH₃CONH | H | H | H | H | H | H | H | H | H | 405 | 1.87 |
| A159 | CH₃NH | sec-Butyl | O | H | CH₃CONH | H | H | H | H | H | H | H | H | H | 419 | 2.08 |
| A160 | CH₃NH | Methyl | O | H | CH₃CONH | H | H | H | H | H | H | H | H | H | 377 | 1.47 |
| A161 | CH₃NH | Isobutyl | O | H | CH₃CONH | H | H | H | H | H | H | H | H | H | 419 | 2.14 |
| A162 | CH₃CH₂CH₂NH | Cyclopropyl- | O | H | CH₃CONH | H | H | H | H | H | H | H | H | H | 445 | 2.54 |
| | | methyl | | | | | | | | | | | | | | |
| A163 | CH₃NH | 2-Methoxy- | O | H | CH₃CONH | H | H | H | H | H | H | H | H | H | 421 | 1.60 |
| | | ethyl | | | | | | | | | | | | | | |
| A164 | CH₃NH | Ethyl | O | H | F | H | H | H | H | H | H | H | H | H | 352 | 2.54 |
| A165 | CH₃NH | Allyl | O | H | F | H | H | H | H | H | H | H | H | H | 364 | 2.78 |
| A166 | CH₃CH₂NH | Propyl | O | H | F | H | H | H | H | H | H | H | H | H | 380 | 3.21 |
| A167 | CH₃NH | Butyl | O | H | F | H | H | H | H | H | H | H | H | H | 380 | 3.25 |
| A168 | CH₃NH | Propyl | O | H | F | H | H | H | H | H | H | H | H | H | 366 | 2.86 |
| A169 | CH₃CH₂NH | Ethyl | O | H | F | H | H | H | H | H | H | H | H | H | 366 | 2.86 |
| A170 | CH₃NH | Cyclopropyl- | O | H | F | H | H | H | H | H | H | H | H | H | 378 | 2.98 |
| | | methyl | | | | | | | | | | | | | | |
| A171 | CH₃NH | Isopropyl | O | H | F | H | H | H | H | H | H | H | H | H | 366 | 2.78 |
| A172 | CH₃NH | sec-Butyl | O | H | F | H | H | H | H | H | H | H | H | H | 380 | 3.11 |
| A173 | CH₃NH | Methyl | O | H | F | H | H | H | H | H | H | H | H | H | 338 | 2.25 |
| A174 | CH₃NH | Isobutyl | O | H | F | H | H | H | H | H | H | H | H | H | 380 | 3.17 |
| A175 | Cyclopropyl- | 2-Cyano- | O | H | F | H | H | H | H | H | H | H | H | H | 403 | 2.63 |
| | amino | ethyl | | | | | | | | | | | | | | |
| A176 | CH₃NH | Ethyl | O | H | CHF₂ | H | H | H | H | H | H | H | H | H | 384 | 2.54 |
| A177 | CH₃NH | Allyl | O | H | CHF₂ | H | H | H | H | H | H | H | H | H | 396 | 2.78 |
| A178 | CH₃CH₂NH | Propyl | O | H | CHF₂ | H | H | H | H | H | H | H | H | H | 412 | 3.19 |
| A179 | CH₃NH | Butyl | O | H | CHF₂ | H | H | H | H | H | H | H | H | H | 412 | 3.21 |
| A180 | CH₃NH | Propyl | O | H | CHF₂ | H | H | H | H | H | H | H | H | H | 398 | 2.86 |
| A181 | CH₃CH₂NH | Ethyl | O | H | CHF₂ | H | H | H | H | H | H | H | H | H | 398 | 2.84 |
| A182 | CH₃NH | Cyclopropyl- | O | H | CHF₂ | H | H | H | H | H | H | H | H | H | 410 | 2.96 |
| | | methyl | | | | | | | | | | | | | | |
| A183 | CH₃NH | Isopropyl | O | H | CHF₂ | H | H | H | H | H | H | H | H | H | 398 | 2.77 |
| A184 | CH₃NH | Methyl | O | H | CHF₂ | H | H | H | H | H | H | H | H | H | 370 | 2.25 |
| A185 | CH₃NH | Isobutyl | O | H | CHF₂ | H | H | H | H | H | H | H | H | H | 412 | 3.15 |
| A186 | CH₃CH₂CH₂NH | Cyclopropyl- | O | H | CHF₂ | H | H | H | H | H | H | H | H | H | 438 | 3.64 |
| | | methyl | | | | | | | | | | | | | | |
| A187 | Cyclopropyl- | 2-Cyano- | O | H | CHF₂ | H | H | H | H | H | H | H | H | H | 435 | 2.64 |
| | amino | ethyl | | | | | | | | | | | | | | |
| A188 | CH₃NH | 2-Methoxy- | O | H | CHF₂ | H | H | H | H | H | H | H | H | H | 414 | 2.42 |
| | | ethyl | | | | | | | | | | | | | | |
| A189 | CH₃NH | 3-Methoxy- | O | H | CHF₂ | H | H | H | H | H | H | H | H | H | 428 | 2.49 |
| | | propyl | | | | | | | | | | | | | | |
| A190 | CH₃NH | Ethyl | O | H | CF₃O | H | H | H | H | H | H | H | H | H | 418 | 3.15 |
| A191 | CH₃NH | Allyl | O | H | CF₃O | H | H | H | H | H | H | H | H | H | 430 | 3.42 |
| A192 | CH₃CH₂NH | Propyl | O | H | CF₃O | H | H | H | H | H | H | H | H | H | 446 | 3.89 |
| A193 | CH₃NH | Butyl | O | H | CF₃O | H | H | H | H | H | H | H | H | H | 446 | 3.89 |
| A194 | CH₃NH | Propyl | O | H | CF₃O | H | H | H | H | H | H | H | H | H | 432 | 3.51 |
| A195 | CH₃CH₂NH | Ethyl | O | H | CF₃O | H | H | H | H | H | H | H | H | H | 432 | 3.51 |
| A196 | CH₃NH | Cyclopropyl- | O | H | CF₃O | H | H | H | H | H | H | H | H | H | 444 | 3.60 |
| | | methyl | | | | | | | | | | | | | | |
| A197 | CH₃NH | Isopropyl | O | H | CF₃O | H | H | H | H | H | H | H | H | H | 432 | 3.42 |
| A198 | CH₃NH | Methyl | O | H | CF₃O | H | H | H | H | H | H | H | H | H | 404 | 2.82 |
| A199 | CH₃NH | Isobutyl | O | H | CF₃O | H | H | H | H | H | H | H | H | H | 446 | 3.85 |
| A200 | CH₃CH₂CH₂NH | Cyclopropyl- | O | H | CF₃O | H | H | H | H | H | H | H | H | H | 472 | 4.38 |
| | | methyl | | | | | | | | | | | | | | |
| A201 | Cyclopropyl- | 2-Cyano- | O | H | CF₃O | H | H | H | H | H | H | H | H | H | 469 | 3.23 |
| | amino | ethyl | | | | | | | | | | | | | | |
| A202 | CH₃NH | 2-Methoxy- | O | H | CF₃O | H | H | H | H | H | H | H | H | H | 448 | 3.02 |
| | | ethyl | | | | | | | | | | | | | | |
| A203 | CH₃NH | 3-Methoxy- | O | H | CF₃O | H | H | H | H | H | H | H | H | H | 462 | 3.09 |
| | | propyl | | | | | | | | | | | | | | |
| A204 | CH₃NH | Ethyl | O | H | CHF₂CF₂O | H | H | H | H | H | H | H | H | H | 450 | 3.06 |
| A205 | CH₃NH | Allyl | O | H | CHF₂CF₂O | H | H | H | H | H | H | H | H | H | 462 | 3.29 |
| A206 | CH₃CH₂NH | Propyl | O | H | CHF₂CF₂O | H | H | H | H | H | H | H | H | H | 478 | 3.80 |
| A207 | CH₃NH | Butyl | O | H | CHF₂CF₂O | H | H | H | H | H | H | H | H | H | 478 | 3.73 |
| A208 | CH₃NH | Propyl | O | H | CHF₂CF₂O | H | H | H | H | H | H | H | H | H | 464 | 3.37 |
| A209 | CH₃CH₂NH | Ethyl | O | H | CHF₂CF₂O | H | H | H | H | H | H | H | H | H | 464 | 3.39 |
| A210 | CH₃NH | Cyclopropyl- | O | H | CHF₂CF₂O | H | H | H | H | H | H | H | H | H | 476 | 3.46 |
| | | methyl | | | | | | | | | | | | | | |
| A211 | CH₃NH | Isopropyl | O | H | CHF₂CF₂O | H | H | H | H | H | H | H | H | H | 464 | 3.29 |
| A212 | CH₃NH | Methyl | O | H | CHF₂CF₂O | H | H | H | H | H | H | H | H | H | 436 | 2.75 |
| A213 | CH₃NH | Isobutyl | O | H | CHF₂CF₂O | H | H | H | H | H | H | H | H | H | 478 | 3.69 |
| A214 | CH₃CH₂CH₂NH | Cyclopropyl- | O | H | CHF₂CF₂O | H | H | H | H | H | H | H | H | H | 504 | 4.17 |
| | | methyl | | | | | | | | | | | | | | |
| A215 | Cyclopropyl- | 2-Cyano- | O | H | CHF₂CF₂O | H | H | H | H | H | H | H | H | H | 501 | 3.11 |
| | amino | ethyl | | | | | | | | | | | | | | |
| A216 | CH₃NH | 2-Methoxy- | O | H | CHF₂CF₂O | H | H | H | H | H | H | H | H | H | 480 | 2.94 |
| | | ethyl | | | | | | | | | | | | | | |
| A217 | CH₃NH | 3-Methoxy- | O | H | CHF₂CF₂O | H | H | H | H | H | H | H | H | H | 494 | 3.00 |
| | | propyl | | | | | | | | | | | | | | |
| A218 | CH₃NH | Ethyl | O | H | F | H | H | F | H | H | H | H | H | H | 370 | 2.66 |
| A219 | CH₃NH | Allyl | O | H | F | H | H | F | H | H | H | H | H | H | 382 | 2.92 |
| A220 | CH₃CH₂NH | Propyl | O | H | F | H | H | F | H | H | H | H | H | H | 398 | 3.37 |
| A221 | CH₃NH | Butyl | O | H | F | H | H | F | H | H | H | H | H | H | 398 | 3.39 |
| A222 | CH₃NH | Propyl | O | H | F | H | H | F | H | H | H | H | H | H | 384 | 3.00 |
| A223 | CH₃CH₂NH | Ethyl | O | H | F | H | H | F | H | H | H | H | H | H | 384 | 3.00 |
| A224 | CH₃NH | Cyclopropyl- | O | H | F | H | H | F | H | H | H | H | H | H | 396 | 3.11 |
| | | methyl | | | | | | | | | | | | | | |
| A225 | CH₃NH | Isopropyl | O | H | F | H | H | F | H | H | H | H | H | H | 384 | 2.92 |
| A226 | CH₃NH | Methyl | O | H | F | H | H | F | H | H | H | H | H | H | 356 | 2.35 |
| A227 | CH₃NH | Isobutyl | O | H | F | H | H | F | H | H | H | H | H | H | 398 | 3.31 |
| A228 | CH₃CH₂CH₂NH | Cyclopropyl- | O | H | F | H | H | F | H | H | H | H | H | H | 424 | 3.85 |
| | | methyl | | | | | | | | | | | | | | |
| A229 | Cyclopropyl- | 2-Cyano- | O | H | F | H | H | F | H | H | H | H | H | H | 421 | 2.73 |
| | amino | ethyl | | | | | | | | | | | | | | |
| A230 | CH₃NH | 2-Methoxy- | O | H | F | H | H | F | H | H | H | H | H | H | 400 | 2.52 |
| | | ethyl | | | | | | | | | | | | | | |
| A231 | CH₃NH | 3-Methoxy- | O | H | F | H | H | F | H | H | H | H | H | H | 414 | 2.61 |
| | | propyl | | | | | | | | | | | | | | |
| A232 | CH₃NH | Ethyl | O | H | Cl | H | H | F | H | H | H | H | H | H | 386 | 2.94 |
| A233 | CH₃NH | Allyl | O | H | Cl | H | H | F | H | H | H | H | H | H | 398 | 3.21 |
| A234 | CH₃CH₂NH | Propyl | O | H | Cl | H | H | F | H | H | H | H | H | H | 414 | 3.69 |
| A235 | CH₃NH | Butyl | O | H | Cl | H | H | F | H | H | H | H | H | H | 414 | 3.71 |
| A236 | CH₃NH | Propyl | O | H | Cl | H | H | F | H | H | H | H | H | H | 400 | 3.29 |
| A237 | CH₃CH₂NH | Ethyl | O | H | Cl | H | H | F | H | H | H | H | H | H | 400 | 3.29 |
| A238 | CH₃NH | Cyclopropyl- | O | H | Cl | H | H | F | H | H | H | H | H | H | 412 | 3.39 |
| | | methyl | | | | | | | | | | | | | | |
| A239 | CH₃NH | Isopropyl | O | H | Cl | H | H | F | H | H | H | H | H | H | 400 | 3.21 |
| A240 | CH₃NH | Methyl | O | H | Cl | H | H | F | H | H | H | H | H | H | 372 | 2.61 |
| A241 | CH₃NH | Isobutyl | O | H | Cl | H | H | F | H | H | H | H | H | H | 414 | 3.64 |
| A242 | CH₃CH₂CH₂NH | Cyclopropyl- | O | H | Cl | H | H | F | H | H | H | H | H | H | 440 | 4.20 |
| | | methyl | | | | | | | | | | | | | | |
| A243 | Cyclopropyl- | 2-Cyano- | O | H | Cl | H | H | F | H | H | H | H | H | H | 437 | 3.02 |
| | amino | ethyl | | | | | | | | | | | | | | |
| A244 | CH₃NH | 2-Methoxy- | O | H | Cl | H | H | F | H | H | H | H | H | H | 416 | 2.78 |
| | | ethyl | | | | | | | | | | | | | | |
| A245 | CH₃NH | 3-Methoxy- | O | H | Cl | H | H | F | H | H | H | H | H | H | 430 | 2.86 |
| | | propyl | | | | | | | | | | | | | | |
| A246 | CH₃NH | Ethyl | O | H | H | 2-Oxo- | H | H | H | H | H | H | H | H | 416 | 1.86 |
| | | | | | | pyrrolidin- | | | | | | | | | | |
| | | | | | | 1-yl | | | | | | | | | | |
| A247 | CH₃NH | Butyl | O | H | H | 2-Oxo- | H | H | H | H | H | H | H | H | 444 | 2.39 |
| | | | | | | pyrrolidin- | | | | | | | | | | |
| | | | | | | 1-yl | | | | | | | | | | |
| A248 | CH₃NH | Propyl | O | H | H | 2-Oxo- | H | H | H | H | H | H | H | H | 430 | 2.10 |
| | | | | | | pyrrolidin- | | | | | | | | | | |
| | | | | | | 1-yl | | | | | | | | | | |
| A249 | CH₃CH₂NH | Ethyl | O | H | H | 2-Oxo- | H | H | H | H | H | H | H | H | 430 | 2.08 |
| | | | | | | pyrrolidin- | | | | | | | | | | |
| | | | | | | 1-yl | | | | | | | | | | |
| A250 | CH₃NH | Methyl | O | H | H | 2-Oxo- | H | H | H | H | H | H | H | H | 402 | 1.65 |
| | | | | | | pyrrolidin- | | | | | | | | | | |
| | | | | | | 1-yl | | | | | | | | | | |
| A251 | CH₃NH | 2-Methyl- | O | H | H | 2-Oxo- | H | H | H | H | H | H | H | H | 446 | 1.78 |
| | | ethyl | | | | pyrrolidin- | | | | | | | | | | |
| | | | | | | 1-yl | | | | | | | | | | |
| A252 | CH₃NH | Ethyl | O | H | CH₃S | H | H | H | H | H | H | H | H | H | 379 | 2.70 |
| A253 | CH₃CH₂NH | Propyl | O | H | CH₃S | H | H | H | H | H | H | H | H | H | 407 | 3.42 |
| A254 | CH₃NH | Butyl | O | H | CH₃S | H | H | H | H | H | H | H | H | H | 407 | 3.44 |
| A255 | CH₃NH | 2-Cyano- | O | H | CH₃S | H | H | H | H | H | H | H | H | H | 404 | 2.51 |
| | | ethyl | | | | | | | | | | | | | | |
| A256 | CH₃NH | Propyl | O | H | CH₃S | H | H | H | H | H | H | H | H | H | 393 | 3.04 |
| A257 | CH₃CH₂NH | Ethyl | O | H | CH₃S | H | H | H | H | H | H | H | H | H | 393 | 3.04 |
| A258 | CH₃NH | Methyl | O | H | CH₃S | H | H | H | H | H | H | H | H | H | 365 | 2.40 |
| A259 | CH₃NH | 2-Methoxy- | O | H | CH₃S | H | H | H | H | H | H | H | H | H | 409 | 2.58 |
| | | ethyl | | | | | | | | | | | | | | |
| A260 | CH₃NH | Ethyl | O | H | Isopropyl | H | H | H | H | H | H | H | H | H | 375 | 3.23 |
| A261 | CH₃CH₂NH | Propyl | O | H | Isopropyl | H | H | H | H | H | H | H | H | H | 403 | 4.04 |
| A262 | CH₃NH | Butyl | O | H | Isopropyl | H | H | H | H | H | H | H | H | H | 403 | 4.04 |
| A263 | CH₃NH | 2-Cyano- | O | H | Isopropyl | H | H | H | H | H | H | H | H | H | 400 | 2.98 |
| | | ethyl | | | | | | | | | | | | | | |
| A264 | CH₃NH | Propyl | O | H | Isopropyl | H | H | H | H | H | H | H | H | H | 389 | 3.62 |
| A265 | CH₃CH₂NH | Ethyl | O | H | Isopropyl | H | H | H | H | H | H | H | H | H | 389 | 3.62 |
| A266 | CH₃NH | Methyl | O | H | Isopropyl | H | H | H | H | H | H | H | H | H | 361 | 2.86 |
| A267 | CH₃NH | 2-Methoxy- | O | H | Isopropyl | H | H | H | H | H | H | H | H | H | 405 | 3.06 |
| | | ethyl | | | | | | | | | | | | | | |
| A268 | CH₃NH | Ethyl | O | H | Cl | H | Cl | H | H | H | H | H | H | H | 401 | 3.53 |
| A269 | CH₃CH₂NH | Propyl | O | H | Cl | H | Cl | H | H | H | H | H | H | H | 429 | 4.39 |
| A270 | CH₃NH | Butyl | O | H | Cl | H | Cl | H | H | H | H | H | H | H | 429 | 4.39 |
| A271 | CH₃NH | 2-Cyano- | O | H | Cl | H | Cl | H | H | H | H | H | H | H | 426 | 3.25 |
| | | ethyl | | | | | | | | | | | | | | |
| A272 | CH₃NH | Propyl | O | H | Cl | H | Cl | H | H | H | H | H | H | H | 415 | 3.94 |
| A273 | CH₃CH₂NH | Ethyl | O | H | Cl | H | Cl | H | H | H | H | H | H | H | 415 | 3.94 |
| A274 | CH₃NH | Methyl | O | H | Cl | H | Cl | H | H | H | H | H | H | H | 387 | 3.19 |
| A275 | CH₃NH | 2-Methoxy- | O | H | Cl | H | Cl | H | H | H | H | H | H | H | 431 | 3.35 |
| | | ethyl | | | | | | | | | | | | | | |
| A276 | CH₃NH | Ethyl | O | H | CH₃OCH₂ | H | H | H | H | H | H | H | H | H | 377 | 2.26 |
| A277 | CH₃CH₂NH | Propyl | O | H | CH₃OCH₂ | H | H | H | H | H | H | H | H | H | 405 | 2.92 |
| A278 | CH₃NH | Butyl | O | H | CH₃OCH₂ | H | H | H | H | H | H | H | H | H | 405 | 2.94 |
| A279 | CH₃NH | 2-Cyano- | O | H | CH₃OCH₂ | H | H | H | H | H | H | H | H | H | 402 | 2.11 |
| | | ethyl | | | | | | | | | | | | | | |
| A280 | CH₃NH | Propyl | O | H | CH₃OCH₂ | H | H | H | H | H | H | H | H | H | 391 | 2.58 |
| A281 | CH₃CH₂NH | Ethyl | O | H | CH₃OCH₂ | H | H | H | H | H | H | H | H | H | 391 | 2.58 |
| A282 | CH₃NH | Methyl | O | H | CH₃OCH₂ | H | H | H | H | H | H | H | H | H | 363 | 2.01 |
| A283 | CH₃NH | 2-Methoxy- | O | H | CH₃OCH₂ | H | H | H | H | H | H | H | H | H | 407 | 2.15 |
| | | ethyl | | | | | | | | | | | | | | |
| A284 | CH₃NH | Ethyl | O | H | CH₃CH₂O | H | H | H | H | H | H | H | H | H | 377 | 2.68 |
| A285 | CH₃CH₂NH | Propyl | O | H | CH₃CH₂O | H | H | H | H | H | H | H | H | H | 405 | 3.39 |
| A286 | CH₃NH | Butyl | O | H | CH₃CH₂O | H | H | H | H | H | H | H | H | H | 405 | 3.42 |
| A287 | CH₃NH | 2-Cyano- | O | H | CH₃CH₂O | H | H | H | H | H | H | H | H | H | 402 | 2.49 |
| | | ethyl | | | | | | | | | | | | | | |
| A288 | CH₃NH | Propyl | O | H | CH₃CH₂O | H | H | H | H | H | H | H | H | H | 391 | 3.02 |
| A289 | CH₃CH₂NH | Ethyl | O | H | CH₃CH₂O | H | H | H | H | H | H | H | H | H | 391 | 3.02 |
| A290 | CH₃NH | Methyl | O | H | CH₃CH₂O | H | H | H | H | H | H | H | H | H | 363 | 2.37 |
| A291 | CH₃NH | 2-Methoxy- | O | H | CH₃CH₂O | H | H | H | H | H | H | H | H | H | 407 | 2.56 |
| | | ethyl | | | | | | | | | | | | | | |
| A292 | CH₃NH | Ethyl | O | H | 3-Oxo- | H | H | H | H | H | H | H | H | H | 432 | 1.66 |
| | | | | | morpholin- | | | | | | | | | | | |
| | | | | | 4-yl | | | | | | | | | | | |
| A293 | CH₃CH₂NH | Propyl | O | H | 3-Oxo- | H | H | H | H | H | H | H | H | H | 460 | 2.14 |
| | | | | | morpholin- | | | | | | | | | | | |
| | | | | | 4-yl | | | | | | | | | | | |
| A294 | CH₃NH | Butyl | O | H | 3-Oxo- | H | H | H | H | H | H | H | H | H | 460 | 2.17 |
| | | | | | morpholin- | | | | | | | | | | | |
| | | | | | 4-yl | | | | | | | | | | | |
| A295 | CH₃NH | 2-Cyano- | O | H | 3-Oxo- | H | H | H | H | H | H | H | H | H | 457 | 1.53 |
| | | ethyl | | | morpholin- | | | | | | | | | | | |
| | | | | | 4-yl | | | | | | | | | | | |
| A296 | CH₃NH | Propyl | O | H | 3-Oxo- | H | H | H | H | H | H | H | H | H | 446 | 1.88 |
| | | | | | morpholin- | | | | | | | | | | | |
| | | | | | 4-yl | | | | | | | | | | | |
| A297 | CH₃CH₂NH | Ethyl | O | H | 3-Oxo- | H | H | H | H | H | H | H | H | H | 446 | 1.88 |
| | | | | | morpholin- | | | | | | | | | | | |
| | | | | | 4-yl | | | | | | | | | | | |
| A298 | CH₃NH | Methyl | O | H | 3-Oxo- | H | H | H | H | H | H | H | H | H | 418 | 1.44 |
| | | | | | morpholin | | | | | | | | | | | |
| | | | | | 4-yl | | | | | | | | | | | |
| A299 | CH₃NH | 2-Methoxy- | O | H | 3-Oxo- | H | H | H | H | H | H | H | H | H | 462 | 1.59 |
| | | ethyl | | | morpholin- | | | | | | | | | | | |
| | | | | | 4-yl | | | | | | | | | | | |
| A300 | CH₃NH | Ethyl | O | H | H | CH₃O | H | H | H | H | H | H | H | H | 363 | 2.20 |
| A301 | CH₃CH₂NH | Propyl | O | H | H | CH₃O | H | H | H | H | H | H | H | H | 391 | 2.82 |
| A302 | CH₃NH | Butyl | O | H | H | CH₃O | H | H | H | H | H | H | H | H | 391 | 2.86 |
| A303 | CH₃NH | 2-Cyano- | O | H | H | CH₃O | H | H | H | H | H | H | H | H | 388 | 2.05 |
| | | ethyl | | | | | | | | | | | | | | |
| A304 | CH₃NH | Propyl | O | H | H | CH₃O | H | H | H | H | H | H | H | H | 377 | 2.51 |
| A305 | CH₃CH₂NH | Ethyl | O | H | H | CH₃O | H | H | H | H | H | H | H | H | 377 | 2.51 |
| A306 | CH₃NH | Methyl | O | H | H | CH₃O | H | H | H | H | H | H | H | H | 349 | 1.94 |
| A307 | CH₃NH | 2-Methoxy- | O | H | H | CH₃O | H | H | H | H | H | H | H | H | 393 | 2.10 |
| | | ethyl | | | | | | | | | | | | | | |
| A308 | CH₃NH | Ethyl | O | H | CH₃O | CH₃O | H | H | H | H | H | H | H | H | 393 | 1.99 |
| A309 | CH₃CH₂NH | Propyl | O | H | CH₃O | CH₃O | H | H | H | H | H | H | H | H | 421 | 2.58 |
| A310 | CH₃CH₂NH | Butyl | O | H | CH₃O | CH₃O | H | H | H | H | H | H | H | H | 421 | 2.61 |
| A311 | CH₃NH | 2-Cyano- | O | H | CH₃O | CH₃O | H | H | H | H | H | H | H | H | 418 | 1.86 |
| | | ethyl | | | | | | | | | | | | | | |
| A312 | CH₃NH | Propyl | O | H | CH₃O | CH₃O | H | H | H | H | H | H | H | H | 407 | 2.26 |
| A313 | CH₃CH₂NH | Ethyl | O | H | CH₃O | CH₃O | H | H | H | H | H | H | H | H | 407 | 2.25 |
| A314 | CH₃NH | Methyl | O | H | CH₃O | CH₃O | H | H | H | H | H | H | H | H | 379 | 1.75 |
| A315 | CH₃NH | 2-Methoxy- | O | H | CH₃O | CH₃O | H | H | H | H | H | H | H | H | 423 | 1.90 |
| | | ethyl | | | | | | | | | | | | | | |
| A316 | CH₃NH | Ethyl | O | H | CH₃O | Cl | H | H | H | H | H | H | H | H | 397 | 2.75 |
| A317 | CH₃CH₂NH | Propyl | O | H | CH₃O | Cl | H | H | H | H | H | H | H | H | 425 | 3.46 |
| A318 | CH₃NH | Butyl | O | H | CH₃O | Cl | H | H | H | H | H | H | H | H | 425 | 3.48 |
| A319 | CH₃NH | 2-Cyano- | O | H | CH₃O | Cl | H | H | H | H | H | H | H | H | 422 | 2.56 |
| | | ethyl | | | | | | | | | | | | | | |
| A320 | CH₃NH | Propyl | O | H | CH₃O | Cl | H | H | H | H | H | H | H | H | 411 | 3.11 |
| A321 | CH₃CH₂NH | Ethyl | O | H | CH₃O | Cl | H | H | H | H | H | H | H | H | 411 | 3.11 |
| A322 | CH₃NH | Methyl | O | H | CH₃O | Cl | H | H | H | H | H | H | H | H | 383 | 2.45 |
| A323 | CH₃NH | 2-Methoxy- | O | H | CH₃O | Cl | H | H | H | H | H | H | H | H | 427 | 2.61 |
| | | ethyl | | | | | | | | | | | | | | |
| A324 | CH₃NH | Allyl | O | H | H | 2-Oxo- | H | H | H | H | H | H | H | H | 428 | 2.04 |
| | | | | | | pyrrolidin- | | | | | | | | | | |
| | | | | | | 1-yl | | | | | | | | | | |
| A325 | CH₃NH | Isopropyl | O | H | H | 2-Oxo- | H | H | H | H | H | H | H | H | 430 | 2.04 |
| | | | | | | pyrrolidin- | | | | | | | | | | |
| | | | | | | 1-yl | | | | | | | | | | |
| A326 | CH₃NH | sec-Butyl | O | H | H | 2-Oxo- | H | H | H | H | H | H | H | H | 444 | 2.26 |
| | | | | | | pyrrolidin- | | | | | | | | | | |
| | | | | | | 1-yl | | | | | | | | | | |
| A327 | CH₃NH | Isobutyl | O | H | H | 2-Oxo- | H | H | H | H | H | H | H | H | 444 | 2.32 |
| | | | | | | pyrrolidin- | | | | | | | | | | |
| | | | | | | 1-yl | | | | | | | | | | |
| A328 | CH₃CH₂CH₂NH | Cyclopropyl- | O | H | H | 2-Oxo- | H | H | H | H | H | H | H | H | 470 | 2.71 |
| | | methyl | | | | pyrrolidin- | | | | | | | | | | |
| | | | | | | 1-yl | | | | | | | | | | |
| A329 | CH₃NH | 3-Methoxy- | O | H | H | 2-Oxo- | H | H | H | H | H | H | H | H | 460 | 1.84 |
| | | propyl | | | | pyrrolidin- | | | | | | | | | | |
| | | | | | | 1-yl | | | | | | | | | | |
| A330 | CH₃CH₂NH | 2-Cyano- | O | H | H | 2-Oxo- | H | H | H | H | H | H | H | H | 455 | 1.93 |
| | | ethyl | | | | pyrrolidin- | | | | | | | | | | |
| | | | | | | 1-yl | | | | | | | | | | |
| A331 | CH₃NH | Allyl | O | H | CH₃S | H | H | H | H | H | H | H | H | H | 391 | 2.96 |
| A332 | CH₃NH | Isopropyl | O | H | CH₃S | H | H | H | H | H | H | H | H | H | 393 | 2.96 |
| A333 | CH₃NH | sec-Butyl | O | H | CH₃S | H | H | H | H | H | H | H | H | H | 407 | 3.29 |
| A334 | CH₃NH | Isobutyl | O | H | CH₃S | H | H | H | H | H | H | H | H | H | 407 | 3.37 |
| A335 | CH₃CH₂CH₂NH | Cyclopropyl- | O | H | CH₃S | H | H | H | H | H | H | H | H | H | 433 | 3.89 |
| | | methyl | | | | | | | | | | | | | | |
| A336 | CH₃NH | 3-Methoxy- | O | H | CH₃S | H | H | H | H | H | H | H | H | H | 423 | 2.64 |
| | | propyl | | | | | | | | | | | | | | |
| A337 | CH₃CH₂NH | 2-Cyano- | O | H | CH₃S | H | H | H | H | H | H | H | H | H | 418 | 2.80 |
| | | ethyl | | | | | | | | | | | | | | |
| A338 | CH₃NH | Allyl | O | H | Isopropyl | H | H | H | H | H | H | H | H | H | 387 | 3.53 |
| A339 | CH₃NH | Isopropyl | O | H | Isopropyl | H | H | H | H | H | H | H | H | H | 389 | 3.51 |
| A340 | CH₃NH | sec-Butyl | O | H | Isopropyl | H | H | H | H | H | H | H | H | H | 403 | 3.92 |
| A341 | CH₃NH | Isobutyl | O | H | Isopropyl | H | H | H | H | H | H | H | H | H | 403 | 3.96 |
| A342 | CH₃CH₂CH₂NH | Cyclopropyl- | O | H | Isopropyl | H | H | H | H | H | H | H | H | H | 429 | 4.51 |
| | | methyl | | | | | | | | | | | | | | |
| A343 | CH₃NH | 3-Methoxy- | O | H | Isopropyl | H | H | H | H | H | H | H | H | H | 419 | 3.15 |
| | | propyl | | | | | | | | | | | | | | |
| A344 | CH₃CH₂NH | 2-Cyano- | O | H | Isopropyl | H | H | H | H | H | H | H | H | H | 414 | 3.31 |
| | | ethyl | | | | | | | | | | | | | | |
| A345 | CH₃NH | Allyl | O | H | Cl | H | Cl | H | H | H | H | H | H | H | 413 | 3.85 |
| A346 | CH₃NH | Isopropyl | O | H | Cl | H | Cl | H | H | H | H | H | H | H | 415 | 3.85 |
| A347 | CH₃NH | sec-Butyl | O | H | Cl | H | Cl | H | H | H | H | H | H | H | 429 | 4.26 |
| A348 | CH₃NH | Isobutyl | O | H | Cl | H | Cl | H | H | H | H | H | H | H | 429 | 4.31 |
| A349 | CH₃CH₂CH₂NH | Cyclopropyl- | O | H | Cl | H | Cl | H | H | H | H | H | H | H | 455 | 4.89 |
| | | methyl | | | | | | | | | | | | | | |
| A350 | CH₃NH | 3-Methoxy- | O | H | Cl | H | Cl | H | H | H | H | H | H | H | 445 | 3.44 |
| | | propyl | | | | | | | | | | | | | | |
| A351 | CH₃CH₂NH | 2-Cyano- | O | H | Cl | H | Cl | H | H | H | H | H | H | H | 440 | 3.60 |
| | | ethyl | | | | | | | | | | | | | | |
| A352 | CH₃NH | Allyl | O | H | CH₃OCH₂ | H | H | H | H | H | H | H | H | H | 389 | 2.51 |
| A353 | CH₃NH | Isopropyl | O | H | CH₃OCH₂ | H | H | H | H | H | H | H | H | H | 391 | 2.51 |
| A354 | CH₃NH | sec-Butyl | O | H | CH₃OCH₂ | H | H | H | H | H | H | H | H | H | 405 | 2.80 |
| A355 | CH₃NH | Isobutyl | O | H | CH₃OCH₂ | H | H | H | H | H | H | H | H | H | 405 | 2.86 |
| A356 | CH₃CH₂CH₂NH | Cyclopropyl- | O | H | CH₃OCH₂ | H | H | H | H | H | H | H | H | H | 431 | 3.37 |
| | | methyl | | | | | | | | | | | | | | |
| A357 | CH₃NH | 3-Methoxy- | O | H | CH₃OCH₂ | H | H | H | H | H | H | H | H | H | 421 | 2.23 |
| | | propyl | | | | | | | | | | | | | | |
| A358 | CH₃CH₂CH₂NH | 2-Cyano- | O | H | CH₃OCH₂ | H | H | H | H | H | H | H | H | H | 416 | 2.37 |
| | | ethyl | | | | | | | | | | | | | | |
| A359 | CH₃NH | Allyl | O | H | CH₃CH₂O | H | H | H | H | H | H | H | H | H | 389 | 2.94 |
| A360 | CH₃NH | Isopropyl | O | H | CH₃CH₂O | H | H | H | H | H | H | H | H | H | 391 | 2.94 |
| A361 | CH₃NH | sec-Butyl | O | H | CH₃CH₂O | H | H | H | H | H | H | H | H | H | 405 | 3.27 |
| A362 | CH₃NH | Isobutyl | O | H | CH₃CH₂O | H | H | H | H | H | H | H | H | H | 405 | 3.33 |
| A363 | CH₃CH₂CH₂NH | Cyclopropyl- | O | H | CH₃CH₂O | H | H | H | H | H | H | H | H | H | 431 | 3.87 |
| | | methyl | | | | | | | | | | | | | | |
| A364 | CH₃NH | 3-Methoxy- | O | H | CH₃CH₂O | H | H | H | H | H | H | H | H | H | 421 | 2.63 |
| | | propyl | | | | | | | | | | | | | | |
| A365 | CH₃NH | Allyl | O | H | 3-Oxo- | H | H | H | H | H | H | H | H | H | 444 | 1.84 |
| | | | | | morpholin- | | | | | | | | | | | |
| | | | | | 4-yl | | | | | | | | | | | |
| A366 | CH₃NH | sec-Butyl | O | H | 3-Oxo- | H | H | H | H | H | H | H | H | H | 460 | 2.07 |
| | | | | | morpholin- | | | | | | | | | | | |
| | | | | | 4-yl | | | | | | | | | | | |
| A367 | CH₃NH | Isobutyl | O | H | 3-Oxo- | H | H | H | H | H | H | H | H | H | 460 | 2.11 |
| | | | | | morpholin- | | | | | | | | | | | |
| | | | | | 4-yl | | | | | | | | | | | |
| A368 | CH₃NH | 3-Methoxy- | O | H | 3-Oxo- | H | H | H | H | H | H | H | H | H | 476 | 1.65 |
| | | propyl | | | morpholin- | | | | | | | | | | | |
| | | | | | 4-yl | | | | | | | | | | | |
| A369 | CH₃CH₂CH₂NH | 2-Cyano- | O | H | 3-Oxo- | H | H | H | H | H | H | H | H | H | 471 | 1.74 |
| | | ethyl | | | morpholin- | | | | | | | | | | | |
| | | | | | 4-yl | | | | | | | | | | | |
| A370 | CH₃NH | Allyl | O | H | H | CH₃O | H | H | H | H | H | H | H | H | 375 | 2.44 |
| A371 | CH₃NH | Isopropyl | O | H | H | CH₃O | H | H | H | H | H | H | H | H | 377 | 2.44 |
| A372 | CH₃NH | sec-Butyl | O | H | H | CH₃O | H | H | H | H | H | H | H | H | 391 | 2.71 |
| A373 | CH₃NH | Isobutyl | O | H | H | CH₃O | H | H | H | H | H | H | H | H | 391 | 2.78 |
| A374 | CH₃CH₂CH₂NH | Cyclopropyl- | O | H | H | CH₃O | H | H | H | H | H | H | H | H | 417 | 3.27 |
| | | methyl | | | | | | | | | | | | | | |
| A375 | CH₃NH | 3-Methoxy- | O | H | H | CH₃O | H | H | H | H | H | H | H | H | 407 | 2.18 |
| | | propyl | | | | | | | | | | | | | | |
| A376 | CH₃CH₂NH | 2-Cyano- | O | H | H | CH₃O | H | H | H | H | H | H | H | H | 402 | 2.30 |
| | | ethyl | | | | | | | | | | | | | | |
| A377 | CH₃NH | Allyl | O | H | CH₃O | CH₃O | H | H | H | H | H | H | H | H | 405 | 2.21 |
| A378 | CH₃NH | Isopropyl | O | H | CH₃O | CH₃O | H | H | H | H | H | H | H | H | 407 | 2.21 |
| A379 | CH₃NH | sec-Butyl | O | H | CH₃O | CH₃O | H | H | H | H | H | H | H | H | 421 | 2.49 |
| A380 | CH₃NH | Isobutyl | O | H | CH₃O | CH₃O | H | H | H | H | H | H | H | H | 421 | 2.56 |
| A381 | CH₃CH₂CH₂NH | Cyclopropyl- | O | H | CH₃O | CH₃O | H | H | H | H | H | H | H | H | 447 | 3.00 |
| | | methyl | | | | | | | | | | | | | | |
| A382 | CH₃NH | 3-Methoxy- | O | H | CH₃O | CH₃O | H | H | H | H | H | H | H | H | 437 | 1.99 |
| | | propyl | | | | | | | | | | | | | | |
| A383 | CH₃CH₂NH | 2-Cyano- | O | H | CH₃O | CH₃O | H | H | H | H | H | H | H | H | 432 | 2.11 |
| | | ethyl | | | | | | | | | | | | | | |
| A384 | CH₃NH | Allyl | O | H | CH₃O | Cl | H | H | H | H | H | H | H | H | 409 | 3.02 |
| A385 | CH₃NH | Isopropyl | O | H | CH₃O | Cl | H | H | H | H | H | H | H | H | 411 | 3.02 |
| A386 | CH₃NH | sec-Butyl | O | H | CH₃O | Cl | H | H | H | H | H | H | H | H | 425 | 3.35 |
| A387 | CH₃NH | Isobutyl | O | H | CH₃O | Cl | H | H | H | H | H | H | H | H | 425 | 3.42 |
| A388 | CH₃CH₂CH₂NH | Cyclopropyl- | O | H | CH₃O | Cl | H | H | H | H | H | H | H | H | 451 | 3.92 |
| | | methyl | | | | | | | | | | | | | | |
| A389 | CH₃NH | 3-Methoxy- | O | H | CH₃O | Cl | H | H | H | H | H | H | H | H | 441 | 2.71 |
| | | propyl | | | | | | | | | | | | | | |
| A390 | CH₃CH₂NH | 2-Cyano- | O | H | CH₃O | Cl | H | H | H | H | H | H | H | H | 436 | 2.84 |
| | | ethyl | | | | | | | | | | | | | | |
| A391 | CH₃CH₂NH | Ethyl | S | H | Cl | H | H | H | H | H | H | H | H | H | 398 | 3.19 |

TABLE B

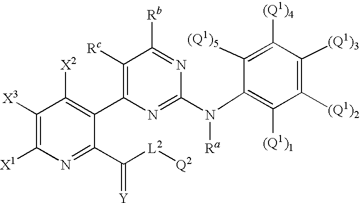

| Example | | | | | | | | | | | | | | | Measured |
|---|---|---|---|---|---|---|---|---|---|---|---|---|---|---|---|
| Number | L² | Q² | Y | (Q₁)¹ | (Q₁)² | (Q₁)³ | (Q₁)⁴ | (Q₁)⁵ | X¹ | X² | X³ | R^{a} | R^{b} | R^{c} | MW |
| B1 | O | isopropyl | O | H | Cl | H | H | H | H | H | H | H | H | H | 369 |
| B2 | O | 2-methoxyethyl | O | H | Cl | H | H | H | H | H | H | H | H | H | 385 |
| B3 | O | pentyl | O | H | Cl | H | H | H | H | H | H | H | H | H | 397 |
| B4 | O | ethyl | O | H | Cl | H | H | H | H | H | H | H | H | H | 355 |
| B5 | O | butyl | O | H | Cl | H | H | H | H | H | H | H | H | H | 383 |
| B6 | O | 3-methoxypropyl | O | H | Cl | H | H | H | H | H | H | H | H | H | 399 |

TABLE B

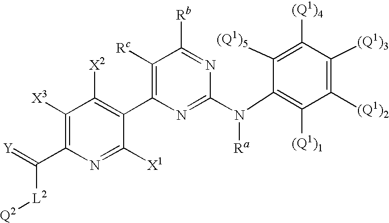

| Example | | | | | | | | | | | | | | | Measured |
|---|---|---|---|---|---|---|---|---|---|---|---|---|---|---|---|
| Number | L² | Q² | Y | (Q₁)¹ | (Q₁)² | (Q₁)³ | (Q₁)⁴ | (Q₁)⁵ | X¹ | X² | X³ | R^{a} | R^{b} | R^{c} | MW |
| C1 | NH | 2-methoxyethyl | O | H | Cl | H | H | H | H | H | H | H | H | H | 383 |

The following examples illustrate in a non-limiting manner the preparation and efficacy of the compounds of formula (I) according to the invention.

### Preparation of 4-{2-[(3-chlorophenyl)amino]pyrimidin-4-yl}-N-[3-(2-oxoazepan-1-yl)propyl]pyridine-2-carboxamide (Compound A-1) according to process P1A

200 mg of N-(3-chlorophenyl)-4-(2-chloropyridin-4-yl)pyrimidin-2-amine (0.63 mmol), 332 mg of 1-(3-aminopropyl)azepan-2-one (1.89 mmol), 166 mg of molybdenum hexacarbonyl (0.631 mmol), 0.282 ml of 1,8-Diazabicyclo(5.4.0)undec-7-ene (1.89 mmol) and 72.9 mg (0.063 mmol) of Tetrakis(triphenylphosphine)palladium(0) were diluted in 3 ml of N,N-dimethylformamide. The reaction mixture was stirred at 100° C. for 6 hours. After cooling, the reaction mixture was poured into 50 ml of HCl 1 M. The precipitate which formed was filtered, washed with NaOH 1M and then water. The crude product was chromatographed on silica (ethyl acetate/heptane) to yield 0.14 g of 4-{2-[(3-chlorophenyl)amino]pyrimidin-4-yl}-N-[3-(2-oxoazepan-1-yl)propyl]pyridine-2-carboxamide (yield=46%). [M+1]=479.

### Preparation of 1-(4-{2-[(3-chlorophenyl)amino]pyrimidin-4-yl}pyridin-2-yl)ethanone (Compound A-110) according to process P1

To 130 ml of tetrahydrofuran was added 55.7 ml of a 1.4M solution of methylmagnesium bromide in toluene and cooled to 0° C. Then 8 g (26 mmol) of 4-{2-[(3-chlorophenyl)amino]pyrimidin-4-yl}pyridine-2-carbonitrile was added in small portions and stirring was continued for 3 hours at 0° C. After warming to room temperature 120 ml of 1N HCl was added and the mixture was extracted with ethyl acetate. The combined organic phases were dried and evaporated to yield 7.4 g of 1-(4-{2-[(3-chlorophenyl)amino]pyrimidin-4-yl}pyridin-2-yl)ethanone (yield=83%). [M+1]=325.

### Preparation of N-(3-chlorophenyl)-4-{2-[-N-methoxyethanimidoyl]pyridin-4-yl}pyrimidin-2-amine (Compound A-93) according to process P2

200 mg (0.62 mmol) of 1-(4-{2-[(3-chlorophenyl)amino]pyrimidin-4-yl}pyridin-2-yl)ethanone, 103 mg (1.24 mmol) of O-methylhydroxylamine hydrochloride and 104 mg (1.26 mmol) sodium acetate dissolved in 12 ml ethanol were stirred at reflux for 8 hours. After cooling the solvent was evaporated in vacuo and 20 ml of water was added. The solid was filtrated, washed with water and dried to yield 157 mg of N-(3-chlorophenyl)-4-{2-[-N-methoxyethanimidoyl]pyridin-4-yl}pyrimidin-2-amine (yield=65%).

[M+1]=354.

### Preparation of 3-{2-[1-(4-{2-[(3-chlorophenyl)amino]pyrimidin-4-yl}pyridin-2-yl)ethylidene]hydrazinyl}propanenitrile (Compound A-108) according to process P2

To a solution of 200 mg (0.62 mmol) 1-(4-{2-[(3-chlorophenyl)amino]pyrimidin-4-yl}pyridin-2-yl)ethanone, 0.028 (0.49 mmol) acetic acid and 51 mg (0.62 mmol) sodium acetate in 2 ml methanol was added 79 mg (0.92 mmol) of 3-hydrazinylpropanenitrile dissolved in a minimum amount of methanol and stirred at reflux for 2 hours and at room temperature overnight. The solvent was evaporated in vacuo and 30 ml of water was added. The mixture was extracted with ethyl acetate, the combined organic phases were dried and evaporated to yield 210 mg of 3-{2-[1-(4-{2-[(3-chlorophenyl)amino]pyrimidin-4-yl}pyridin-2-yl)ethylidene]hydrazinyl}propanenitrile (yield=78%).

[M+1]=392.

### Preparation of 4-{2-[(3-chlorophenyl)amino]pyrimidin-4-yl}pyridine-2-carboxylic acid-monolithium salt (Compound A-17) according to process P9

1300 mg of ethyl 4-{2-[(3-chlorophenyl)amino]pyrimidin-4-yl}pyridine-2-carboxylate (3.66 mmol), 263 mg of lithium hydroxide (11.0 mmol) were stirred for 6 hours at room temperature in a mixture of 10 ml of tetrahydrofuran and 1 ml of water. The precipitate which formed was filtered, washed with dichloromethane and dried to yield 1120 mg of 4-{2-[(3-chlorophenyl)amino]pyrimidin-4-yl}pyridine-2-carboxylic acid-monolithium salt (yield=80%).

¹H NMR: δ (ppm) DMSO: 10.1 (s, 1H); 8.7 (m, 3H); 8.6 (s, 1H); 8.05 (s, 1H); 7.95 (d, 1H); 7.80 (dd, 1H); 7.55 (d, 1H); 7.35 (t, 1H); 7.05 d, 1H)

### Preparation of 4-{2-[(3-chlorophenyl)amino]pyrimidin-4-yl}-N-(3-methylbutyl)pyridine-2-carboxamide (Compound A-19) according to process P9

1300 mg of ethyl 4-{2-[(3-chlorophenyl)amino]pyrimidin-4-yl}pyridine-2-carboxylate (3.66 mmol), 129 mg of 3-methylbutan-1-amine (1.48 mmol) and 48.7 mg of potassium carbonate (0.35 mmol) were stirred for 5 hours at 50° C. in 2.5 ml of N,N-dimethylformamide. The reaction mixture is poured in 30 ml of water, the precipitate which formed was filtered, dried and purified by LC-MS (acetonitrile/water) to yield 4 mg of 4-{2-[(3-chlorophenyl)amino]pyrimidin-4-yl}-N-(3-methylbutyl)pyridine-2-carboxamide (yield=2%).

[M+1]=396.

### Preparation of 4-{2-[(3-chlorophenyl)amino]pyrimidin-4-yl}-N,N-dimethylpyridine-2-carboxamide (Compound A-76) according to process P9

To 250 mg (0.765 mmol) of 4-{2-[(3-chlorophenyl)amino]pyrimidin-4-yl}pyridine-2-carboxylic acid dissolved in 7 ml dichloromethane was added under an argon atmosphere 0.32 ml of triethylamine (2.3 mmol) and cooled to 0° C. Then 0.19 ml (1.53 mmol) 2,2-dimethylpropionyl chloride was added and stirred for 1 hour at 0° C. After the addition of 1.15 ml of a 2M solution of dimethylamine in tetrahydrofuran stirring was continued for 1 hour at 0° C. and for 3 hours at room temperature. The reaction mixture was washed with water and sodium bicarbonate solution. The aqueous layers were extracted with dichloromethane, the combined organic phases were dried, evaporated and purified by chromatography on silica (ethyl acetate/heptane) to yield 130 mg (yield=48%) of 4-{2-[(3-chlorophenyl)amino]pyrimidin-4-yl}-N,N-dimethylpyridine-2-carboxamide.

[M+1]=354.

### Preparation of 4-{2-[(3-chlorophenyl)amino]pyrimidin-4-yl}-N,N-diethylpyridine-2-carboxamide (Compound A-77) according to process P11

1 g (5.43 mmol) of magnesium bromide was dissolved in 50 ml tetrahydrofuran followed by the addition of 3.85 g (10.86 mmol) of ethyl 4-{2-[(3-chlorophenyl)amino]pyrimidin-4-yl}pyridine-2-carboxylate. After stirring for 5 minutes 2.25 ml (21.72 mmol) diethylamine was added and the mixture was stirred at room temperature for 14 hours and at 40° C. for 6 hours. After cooling to room temperature water was added yielding a precipitate which was filtered, washed with 1N sodium hydroxide, dried and purified by chromatography on silica (ethyl acetate/heptane) to yield 1.7 g (yield=39%) of 4-{2-[(3-chlorophenyl)amino]pyrimidin-4-yl}-N,N-diethylpyridine-2-carboxamide.

[M+1]=382.

### Preparation of 4-{2-[(3-chlorophenyl)amino]pyrimidin-4-yl}-N,N-diethylpyridine-2-carbothioamide (Compound A-391) according to process P10

To 200 mg (0.52 mmol) of 4-{2-[(3-chlorophenyl)amino]pyrimidin-4-yl}-N,N-diethylpyridine-2-carboxamide dissolved in 5 ml toluene was added 58 mg (0.26 mmol) of phosphorous pentasulfide and stirred at 100° C. for 1 hour. After cooling the mixture was extracted twice with saturated bicarbonate solution and brine. The organic layer was dried, evaporated and purified by chromatography on silica (ethyl acetate/heptane) to yield 180 mg (yield=82%) of 4-{2-[(3-chlorophenyl)amino]pyrimidin-4-yl}-N,N-diethylpyridine-2-carbothioamide.

[M+1]=398.

### Preparation of propan-2-yl 3-{2-[(3-chlorophenyl)amino]pyrimidin-4-yl}pyridine-2-carboxylate (Compound B-1) and 2-methoxyethyl 3-{2-[(3-chlorophenyl)amino]pyrimidin-4-yl}pyridine-2-carboxylate (Compound B-2)

310 mg of propan-2-yl 3-[3-(dimethylamino)prop-2-enoyl]pyridine-2-carboxylate (1.18 mmol), 275 mg of 3-chlorophenyl-guanidine nitrate (1.18 mmol) and 125 mg of sodium carbonate (1.18 mmol) were stirred for 8 hours at reflux in 2.25 ml of 2-methoxyethanol. After cooling dichloromethane was added and the remaining precipitate was filtered. The filtrate was evaporated and purified by chromatography on silica (ethyl acetate/heptane) to yield 84 mg of propan-2-yl 3-{2-[(3-chlorophenyl)amino]pyrimidin-4-yl}pyridine-2-carboxylate (yield=18%) [M+1]=369 and 67 mg of 2-methoxyethyl 3-{2-[(3-chlorophenyl)amino]pyrimidin-4-yl}pyridine-2-carboxylate (yield=18%) [M+1]=385.

### Preparation of 5-{2-[(3-chlorophenyl)amino]pyrimidin-4-yl}-N-(2-methoxyethyl)pyridine-2-carboxamide (Compound C-1)

### Step 1

To 8.932 g (57.41 mmol) of 1-(6-chloropyridin-3-yl)ethanone was added 65 ml (379 mmol) of 1,1-diethoxy-N,N-dimethylmethanamine and stirred at 95° C. for 2 hours. After cooling the precipitate was filtered, washed with 20 ml pentane to yield 10.29 g of 1-(6-chloropyridin-3-yl)-3-(dimethylamino)prop-2-en-1-one (yield=83%).

[M+1]=211.

### Step 2

1129 mg of 1-(6-chloropyridin-3-yl)-3-(dimethylamino)prop-2-en-1-one (5.36 mmol), 1247 mg of 3-chlorophenyl-guanidine nitrate (5.36 mmol) and 236 mg of sodium hydroxide (5.89 mmol) dissolved in 11.4 ml of 2-propanol were stirred for 8 hours at reflux. After cooling the solvent was removed in vacuo. To the remaining solid were added dichloromethane and 15 ml water and the remaining precipitate was filtered. After phase separation of the filtrate the aqueous phase was extracted with dichloromethane, the combined organic phase were dried and evaporated. The crude product and the precipitate were purified by chromatography on silica (ethyl acetate/heptane) to yield 353 mg of N-(3-chlorophenyl)-4-(6-chloropyridin-3-yl)pyrimidin-2-amine (yield=17%).

[M+1]=318.

### Step 3

300 mg of N-(3-chlorophenyl)-4-(6-chloropyridin-3-yl)pyrimidin-2-amine (0.95 mmol), 213 mg of 2-methoxyethylamine (2.84 mmol), 250 mg of molybdenum hexacarbonyl (0.95 mmol), 0.424 ml of 1,8-Diazabicyclo(5.4.0)undec-7-ene (2.84 mmol) and 1156 mg (0.095 mmol) of Tetrakis(triphenylphosphine)palladium(0) were diluted in 3.6 ml of N,N-dimethylformamide. The reaction mixture was stirred at 100° C. for 5 hours. After cooling, the reaction mixture was poured into 60 ml of HCl 1 M. The precipitate which formed was filtered, washed with NaOH 1M and then water. The crude product was chromatographed on silica (ethyl acetate/heptane) to yield 30 mg of 5-{2-[(3-chlorophenyl)amino]pyrimidin-4-yl}-N-(2-methoxyethyl)pyridine-2-carboxamide (yield=8%).

[M+1]=384.

### BIOLOGICAL EXAMPLE A

### In Vivo Test on Peronospora parasitica (Crucifer Downy Mildew)

Cabbage plants (Eminence variety) in starter cups, sown on a 50/50 peat soil-pozzolana substrate and grown at 18-20° C., are treated at the cotyledon stage by spraying with the aqueous suspension described above. Plants, used as controls, are treated with an aqueous solution not containing the active material. After 24 hours, the plants are contaminated by spraying them with an aqueous suspension of Peronospora parasitica spores (50 000 spores per ml). The spores are collected from infected plant. The contaminated cabbage plants are incubated for 5 days at 20° C., under a humid atmosphere. Grading is carried out 5 days after the contamination, in comparison with the control plants.

Under these conditions, good protection (at least 70%) is observed at a dose of 500 ppm with the following compounds: A75, A76, A77 and A83.

### BIOLOGICAL EXAMPLE B

### In Vivo Test on Sphaerotheca fuliginea (Cucurbits Powdery Mildew)

To produce a suitable preparation of active compound, 1 part by weight of active compound is mixed with 49 parts by weight of solvent (N,N-dimethylformamide) and 1 part by weight of emulsifier (alkylarylpolyglycolether), and the concentrate is diluted with water to the desired concentration. To test for preventive activity, young cucurbit plants are sprayed with the preparation of active compound at the stated rate of application. One day after this treatment, the plants are inoculated with an aqueous spore suspension of Sphaerotheca fuliginea. Then the plants are placed in a greenhouse at approximately 23° C. and a relative atmospheric humidity of approximately 70%. Grading is carried out 7 days after the contamination, in comparison with the control plants. Under these conditions, good (at least 70%) protection is observed at a dose of 500 ppm with the following compound: A16.

### BIOLOGICAL EXAMPLE C

### In Vivo Test on Sphaerotheca fuliginea (Cucurbits Powdery Mildew)

The active ingredients tested are prepared by homogenization in a mixture of acetone/tween/DMSO, then diluted with water to obtain the desired active material. Gherkin plants (Vert petit de Paris variety) in starter cups, sown on a 50/50 peat soil-pozzolana substrate and grown at 20° C./23° C., are treated at the cotyledon Z10 stage by spraying with the aqueous suspension described above. Plants, used as controls, are treated with an aqueous solution not containing the active material.

After 24 hours, the plants are contaminated by spraying them with an aqueous suspension of Sphaerotheca fuliginea spores (100 000 spores per ml). The spores are collected from a contaminated plants. The contaminated gherkin plants are incubated at about 20° C./25° C. and at 60/70% relative humidity.

Grading (% of efficacy) is carried out 12 days after the contamination, in comparison with the control plants.

Under these conditions, good (at least 70%) or total protection is observed at a dose of 500 ppm with the following compounds: A76, A79 and A83.

### BIOLOGICAL EXAMPLE D

### In Vivo Test on Botrytis cinerea (Grey Mould)

The active ingredients tested are prepared by homogenization in a mixture of acetone/tween/DMSO, then diluted with water to obtain the desired active material.

Gherkin plants (Vert petit de Paris variety), sown on a 50/50 peat soil-pozzolana substrate in starter cups and grown at 18-20° C., are treated at the cotyledon Z11 stage by spraying with the active ingredient prepared as described above.

Plants, used as controls, are treated with an aqueous solution not containing the active material. After 24 hours, the plants are contaminated by depositing drops of an aqueous suspension of Botrytis cinerea spores (150,000 spores per ml) on upper surface of the leaves. The spores are collected from a 15-day-old culture and are suspended in a nutrient solution composed of:
20 g/L of gelatin;
50 g/L of D-fructose;
2 g/L of NH₄NO₃;
1 g/L of KH₂PO₄.

The contaminated cucumber plants are settled for 5-7 days in a climatic room at 15-11° C. (day/night) and at 80% relative humidity.

Grading is carried out 5/7 days after the contamination, in comparison with the control plants. Under these conditions, good (at least 70%) protection is observed at a dose of 500 ppm with the following compounds: A1, A3, A4, A75, A76, A77, A78, A79, A82, A86, A87 and A89.

### BIOLOGICAL EXAMPLE E

### In Vivo Test on Alternaria solani (Tomato Leaf Spot)

To produce a suitable preparation of active compound, 1 part by weight of active compound is mixed with 49 parts by weight of solvent (N,N-dimethylformamide) and 1 part by weight of emulsifier (alkylarylpolyglycolether), and the concentrate is diluted with water to the desired concentration. To test for preventive activity, young tomato plants are sprayed with the preparation of active compound at the stated rate of application. One day after this treatment, the tomato plants are inoculated with an aqueous spore suspension of Alternaria solani. The plants remain for one day in an incubation cabinet at approximately 20° C. and a relative atmospheric humidity of 100%. Then the plants are placed in an incubation cabinet at approximately 20° C. and a relative atmospheric humidity of 96%.

Grading is carried out 7 days after the contamination, in comparison with the control plants.

Under these conditions, good (at least 70%) or total protection is observed at a dose of 500 ppm with the following compounds: A1, A11, A13 and A14.

### BIOLOGICAL EXAMPLE F

### In Vivo Test on Alternaria brassicae (Leaf Spot of Cruciferous Plant)

The active ingredients tested are prepared by homogenization in a mixture of acetone/tween/DMSO, and then diluted with water to obtain the desired active material.

Radish plants (Pernot variety), sown on a 50/50 peat soil-pozzolana substrate in starter cups and grown at 18-20° C., are treated at the cotyledon stage by spraying with the active ingredient prepared as described above.

Plants, used as controls, are treated with the mixture of acetone/tween/water not containing the active material.

After 24 hours, the plants are contaminated by spraying them with an aqueous suspension of Alternaria brassicae spores (40,000 spores per cm³). The spores are collected from a 12 to 13 days-old culture.

The contaminated radish plants are incubated for 6-7 days at about 18° C., under a humid atmosphere.

Grading is carried out 6 to 7 days after the contamination, in comparison with the control plants.

Under these conditions, good protection (at least 70%) is observed at a dose of 500 ppm with the following compounds: A61, A65, A69, A75, A77, A78, A79, A80, A81, A82, A84, A85, A86 and A89.

### BIOLOGICAL EXAMPLE G

### In Vivo Test on Puccinia recondite (Wheat Rust)

To produce a suitable preparation of active compound, 1 part by weight of active compound is mixed with 49 parts by weight of solvent (N,N-dimethylformamide) and 1 part by weight of emulsifier (alkylarylpolyglycolether), and the concentrate is diluted with water to the desired concentration. To test for preventive activity, young wheat plants are sprayed with the preparation of active compound at the stated rate of application. One day after this treatment, the plants are inoculated with an aqueous spore suspension of Puccinia recondite. The plants remain for 48 hours in an incubation cabinet at 20° C. and a relative atmospheric humidity of 100%. Then the plants are placed in a greenhouse at a temperature of approximately 20° C. and a relative atmospheric humidity of approximately 80%.

Grading is carried out 7 days after the contamination, in comparison with the control plants.

Under these conditions, good (at least 70%) or total protection is observed at a dose of 500 ppm with the following compound: A1 and A3.

### BIOLOGICAL EXAMPLE H

### In Vivo Test on Puccinia recondita (Brown Rust)

The active ingredients tested are prepared by homogenization in a mixture of acetone/tween/DMSO, then diluted with water to obtain the desired active material.

Wheat plants (Scipion variety) sown on 50/50 peat soil-pozzolana substrate in starter cups and grown at 12° C., are treated at the 1-leaf stage (10 cm tall) by spraying with the aqueous suspension described above.

Plants, used as controls, are treated with an aqueous solution not containing the active material.

After 24 hours, the plants are contaminated by spraying the leaves with an aqueous suspension of Puccinia recondita spores (100,000 spores per ml). The spores are collected from a 10-day-old contaminated wheat and are suspended in water containing 2.5 ml/l of tween 80 10%. The contaminated wheat plants are incubated for 24 hours at 20° C. and at 100% relative humidity, and then for 10 days at 20° C. and at 70% relative humidity.

Grading is carried out 10 days after the contamination, in comparison with the control plants.

Under these conditions, good (at least 70%) or total protection is observed at a dose of 500 ppm with the following compounds: A65, A75, A76, A77, A79, A82 and A89.

### BIOLOGICAL EXAMPLE I

### In Vivo Test on Mycosphaerella araminicola (Wheat Leaf Spot)

The active ingredients tested are prepared by homogenization in a mixture of acetone/tween/DMSO, then diluted with water to obtain the desired active material concentration. Wheat plants (Scipion variety), sown on a 50/50 peat soil-pozzolana substrate in starter cups and grown at 12° C., are treated at the 1-leaf stage (10 cm tall) by spraying with the aqueous suspension described above. Plants, used as controls, are treated with an aqueous solution not containing the active material.

After 24 hours, the plants are contaminated by spraying them with an aqueous suspension of Mycosphaerella graminicola spores (500 000 spores per ml). The spores are collected from a 7-day-old culture. The contaminated wheat plants are incubated for 72 hours at 18° C. and at 100% relative humidity, and then for 21 to 28 days at 90% relative humidity.

Grading (% of efficacy) is carried out 21 to 28 days after the contamination, in comparison with the control plants.

Under these conditions, good (at least 70%) or total protection is observed at a dose of 500 ppm with the following compounds: A22, A37, A39, A45, A52, A61, A80, A82, A84, A87, A90, A96, A97, A99, A106 and A109.

### BIOLOGICAL EXAMPLE J

### In Vivo Test on Pyrenophora teres (Barley Net Blotch)

To produce a suitable preparation of active compound, 1 part by weight of active compound is mixed with 49 parts by weight of solvent (N,N-dimethylformamide) and 1 part by weight of emulsifier (alkylarylpolyglycolether), and the concentrate is diluted with water to the desired concentration.

To test for preventive activity, young barley plants are sprayed with the preparation of active compound at the stated rate of application. One day after this treatment, the plants are inoculated with an aqueous spore suspension of Pyrenophora teres. The plants remain for 48 hours in an incubation cabinet at 20° C. and a relative atmospheric humidity of 100%. Then the plants are placed in a greenhouse at a temperature of approximately 20° C. and a relative atmospheric humidity of approximately 80%.

Grading is carried out 7 to 9 days after the contamination, in comparison with the control plants.

Under these conditions, good (at least 70%) or total protection is observed at a dose of 500 ppm with the following compounds: A1, A2, A3, A4 and A16.

### BIOLOGICAL EXAMPLE K

### In Vivo Test on Pyrenophora teres (Barley Net Blotch)

The active ingredients tested are prepared by homogenization in a mixture of acetone/tween/DMSO, then diluted with water to obtain the desired active material concentration.

Barley plants (Express variety), sown on a 50/50 peat soil-pozzolana substrate in starter cups and grown at 12° C., are treated at the 1-leaf stage (10 cm tall) by spraying with the active ingredient prepared as described above. Plants, used as controls, are treated with the mixture of acetone/tween/DMSO/water not containing the active material.

After 24 hours, the plants are contaminated by spraying them with an aqueous suspension of Pyrenophora teres spores (12,000 spores per ml). The spores are collected from a 12-day-old culture. The contaminated barley plants are incubated for 24 hours at about 20° C. and at 100% relative humidity, and then for 12 days at 80% relative humidity.

Grading is carried out 12 days after the contamination, in comparison with the control plants.

Under these conditions, good (at least 70%) or total protection is observed at a dose of 500 ppm with the following compounds: A43, A44, A61, A65, A75, A76, A77, A78, A79, A81, A82, A83, A84, A86, A87, A88, A89, A91, A99, A100, A101, A104, A105, A106, A107, A108, A109, A110 and A111.

### BIOLOGICAL EXAMPLE L

### In Vivo Test on Pyricularia drisea (Rice Blast)

The active ingredients tested are prepared by homogenization in a mixture of acetone/tween/DMSO, then diluted with water to obtain the desired active material concentration. Rice plants (Koshihikari variety), sown on a 50/50 peat soil-pozzolana substrate in starter cups and grown at 25° C., are treated at the 2-leaf stage (13-15 cm tall) by spraying with the active ingredient prepared as described above. Plants, used as controls, are treated with the mixture of acetone/tween/DMSO/water not containing the active material.

After 24 hours, the plants are contaminated by spraying them with an aqueous suspension of Pyricularia grisea spores (30,000 spores per ml). The spores are collected from a 17-day-old culture and are suspended in water containing 2.5 g/l of gelatin. The contaminated Rice plants are incubated for 72 hours at about 25° C. and at 100% relative humidity, and then for 3 days at 25° C. at 80% relative humidity during the day and 20% relative humidity during the night.

Grading (% of efficacy) is carried out 6 days after the contamination, in comparison with the control plants.

Under these conditions, good (at least 70%) or total protection is observed at a dose of 500 ppm with the following compounds: A76 and A91.

### BIOLOGICAL EXAMPLE M

### In Vivo Test on Leptosphaeria nodorum (Wheat Glume Blotch)

Solvent: 49 parts by weight of N,N-Dimethylformamide

Emulsifier: 1 part by weight of Alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with a preparation of active compound at the stated rate of application. One day after this treatment, the plants are inoculated with an aqueous spore suspension of Leptosphaeria nodorum. The plants remain for 48 hours in an incubation cabinet at 22° C. and a relative atmospheric humidity of 100%. Then the plants are placed in a greenhouse at a temperature of approximately 22° C. and a relative atmospheric humidity of approximately 90%.

The test is evaluated 7-9 days after the inoculation. 0% means an efficacy which corresponds to that of the control, while an efficacy of 100% means that no disease is observed.

In this test the compounds according to the invention of the following structures showed efficacy of 70% or even higher at a concentration of 500 ppm of active ingredient: A1, A3 and A4.

1. A phenyl-pyrimidinyl-amino derivative of formula (I) 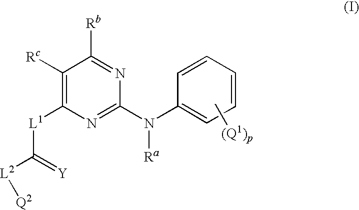
wherein
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-Cₐ-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylsulfanyl, substituted or non-substituted benzylamino, substituted or non-substituted phenoxy, substituted or non-substituted phenylsulfanyl, or substituted or non-substituted phenylamino;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, a C₁-C₈-halogenoalkoxyalkyl having 1 to 5 halogen atoms,
R^{b} and independently represent a hydrogen atom, a halogen atom, a cyano, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms;
L¹ represents a substituted or non substituted pyridyl moiety;
Y represents O, S, NR^{d}, CR^{e}R^{f};
L² represents a direct bond, O, S, NR^{g}, CR^{h}Rⁱ;
Q² represents a hydrogen atom, a halogen atom, a nitro group, a hydroxy group, a cyano group, an amino group, a sulfanyl group, a formyl group, a formyloxy group, a formylamino group, a carbamoyl group, a N-hydroxycarbamoyl group, a carbamate group, substituted or non-substituted (hydroxyimino)-C₁-C₆-alkyl group, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₁₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylsulfanyl, substituted or non-substituted benzylamino, substituted or non-substituted phenoxy, substituted or non-substituted phenylsulfanyl, substituted or non-substituted phenylamino, a substituted or non-substituted or a 4-, 5-, 6- or 7-membered heterocycle comprising up to 4 heteroatoms selected in the list consisting of N, O, S;
L² and Q² can form together a substituted or non-substituted, 4-, 5-, 6- or 7-membered heterocycle comprising up to 4 heteroatoms selected in the list consisting of N, O, S;
R^{d}, R^{e}, R^{f}, R^{g}, R^{h} and Rⁱ independently represent a hydrogen atom, a halogen atom, a nitro group, a cyano group, a hydroxy group, an amino group, a sulfanyl group, a formyl group, a formyloxy group, a formylamino group, a carbamoyl group, a N-hydroxycarbamoyl group, a carbamate group, substituted or non-substituted (hydroxyimino)-C₁-C₆-alkyl group, substituted or non-substituted C₁-C₈-alkyl, tri(C₁-C₈-alkyl)silyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkylcarbamoyl, substituted or non-substituted di-C₁-C₈-alkylcarbamoyl, substituted or non-substituted N—C₁-C₈-alkyloxycarbamoyl, substituted or non-substituted C₁-C₈-alkoxycarbamoyl, substituted or non-substituted N—C₁-C₈-alkyl-C₁-C₈-alkoxycarbamoyl, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted C₁-C₈-alkylsulphinyl, substituted or non-substituted C₁-C₈-halogenoalkylsulphinyl having 1 to 5 halogen atoms, substituted or non-substituted C₁-C₈-alkylsulphonyl, substituted or non-substituted C₁-C₈-halogenoalkylsulphonyl having 1 to 5 halogen atoms, substituted or non-substituted C₁-C₈-alkylaminosulfamoyl, substituted or non-substituted di-C₁-C₈-alkylaminosulfamoyl, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-Cₒ-alkyl, or a 4-, 5-, 6- or 7-membered heterocycle comprising up to 4 heteroatoms selected in the list consisting of N, O, S;
as well as salts, N-oxides, metallic complexes, metalloidic complexes and optically active or geometric isomers thereof; provided that the following compounds are excluded:
5-(2-{[3-(trifluoromethyl)phenyl]amino}pyrimidin-4-yl)nicotinamide;
5-{2-[(tert-butoxycarbonyl)(3-chlorophenyl)amino]-6-methylpyrimidin-4-yl}nicotinic acid;
5-{2-[(tert-butoxycarbonyl)(3-chlorophenyl)amino]pyrimidin-4-yl}nicotinic acid;
ethyl 5-{2-[(3-chlorophenyl)amino]-6-methylpyrimidin-4-yl}nicotinate;
ethyl 5-{2-[tert-butoxycarbonyl)(3-chlorophenyl)amino]-6-methylpyrimidin-4-yl}nicotinate;
4-(2-{[3-(1,1,2,2-tetrafluoroethoxy)phenyl]amino}pyrimidin-4-yl)pyridine-2-carboxamide;
4-(2-{[3-(1,1,2,2-tetrafluoroethoxy)phenyl]amino}pyrimidin-4-yl)pyridine-2-carboxylic acid;
N-(2-hydroxyethyl)-4-(2-{[3-(1,1,2,2-tetrafluoroethoxy)-phenyl]amino}pyrimidin-4-yl)pyridine-2-carboxamide;
N-(3-hydroxypropyl)-4-(2-{[3-(1,1,2,2-tetrafluoroethoxy)-phenyl]amino}pyrimidin-4-yl)pyridine-2-carboxamide;
N-(2-aminoethyl)-4-(2-{[3-(1,1,2,2-tetrafluoroethoxy)-phenyl]amino}pyrimidin-4-yl)pyridine-2-carboxamide hydrochloride;
N-(3-aminopropyl)-4-(2-{[3-(1,1,2,2-tetrafluoroethoxy)-phenyl]amino}pyrimidin-4-yl)pyridine-2-carboxamide hydrochloride;
N-(2-aminoethyl)-4-(2-{[3-(1,1,2,2-tetrafluoroethoxy)-phenyl]amino}pyrimidin-4-yl)pyridine-2-carboxamide;
N-(3-aminopropyl)-4-(2-{[3-(1,1,2,2-tetrafluoroethoxy)-phenyl]amino}pyrimidin-4-yl)pyridine-2-carboxamide;
4-{2-[(3-chlorophenyl)amino]pyrimidin-4-yl}pyridine-2-carboxylic acid;
4-(2-{[3-(trifluoromethyl)phenyl]amino}pyrimidin-4-yl)pyridine-2-carboxylic acid;
4-{2-[(3-chlorophenyl)amino]pyrimidin-4-yl}pyridine-2-carboxamide;
4-(2-{[3-(trifluoromethyl)phenyl]amino}pyrimidin-4-yl)pyridine-2-carboxamide;
4-{2-[(3-chlorophenyl)amino]pyrimidin-4-yl}pyridine-2-carboxylic acid;
N-(2-aminoethyl)-4-{2-[(3-chlorophenyl)amino]pyrimidin-4-yl}pyridine-2-carboxamide hydrochloride;
4-{2-[(3-chlorophenyl)amino]pyrimidin-4-yl}-N-(2-hydroxyethyl)-pyridine-2-carboxamide;
4-{2-[(3-chlorophenyl)amino]pyrimidin-4-yl}-N-(3-hydroxypropyl)-pyridine-2-carboxamide;
N-(3-aminopropyl)-4-{2-[(3-chlorophenyl)amino]pyrimidin-4-yl}pyridine-2-carboxamide hydrochloride;
4-{2-[(3-chlorophenyl)amino]pyrimidin-4-yl}pyridine-2-carboxylic acid-monosodium salt.

2. A compound of formula (I) according to claim 1 wherein Q¹ represents a halogen atom, a nitro group, a hydroxy group, a cyano group, an amino group, a sulfanyl group, a pentafluoro-□⁶-sulfanyl group, a formyl group, a formyloxy group, a formylamino group, a (hydroxyimino)-C₁-C₈-alkyl group, a C₁-C₈-alkyl, a substituted or non-substituted tri(C₁-C₈-alkyl)silyl-C₁-C₈-alkyl, substituted or non-substituted C₁-C₈-cycloalkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₁-C₈-alkylcarbonyl, a C₁-C₈-halogenoalkylcarbonyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a (C₁-C₆-alkoxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms.

3. A compound of formula (I) according to claim 1 wherein p represents 0, 1, 2, or 3.

4. A compound of formula (I) according to claim 1 wherein p represents 1.

5. A compound of formula (I) according to claim 1 wherein R^{a} represents a hydrogen atom or a C₁-C₈-cycloalkyl.

6. A compound of formula (I) according to claim 1 wherein R^{b} and R^{c} independently represent a hydrogen atom, a halogen atom, a cyano, a C₁-C₈-halogenoalkyl having 1 to 5 halogen atoms, a C₁-C₈-halogenocycloalkyl having 1 to 5 halogen atoms.

7. A compound of formula (I) according to claim 1 wherein R^{b} and R^{c} independently represent a hydrogen atom or a halogen atom.

8. A compound of formula (I) according to claim 1 wherein L¹ is selected in the list consisting of: 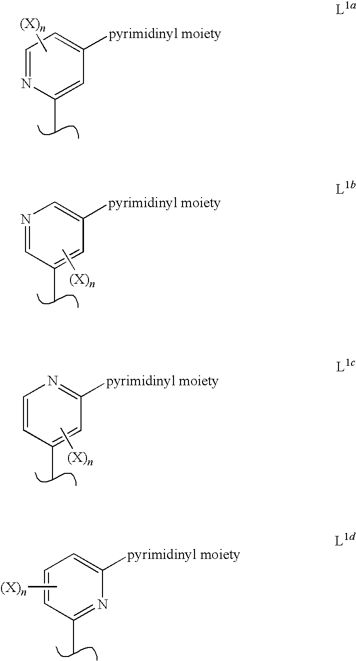
wherein
n represents 0, 1, 2 or 3;
X independently represents a C₁-C₁₀-alkyl, a C₁-C₁₀-halogenoalkyl, a halogen atom or a cyano.

9. A compound of formula (I) according to claim 1 wherein Q² represents a hydrogen atom, a halogen atom, a hydroxy group, a cyano group, an amino group, a sulfanyl group, a formyl group, a formyloxy group, a formylamino group, a carbamoyl group, a N-hydroxycarbamoyl group, a carbamate group, substituted or non-substituted (hydroxyimino)-C₁-C₆-alkyl group, substituted or non-substituted C₁-C₈-alkyl, substituted or non-substituted C₁-C₈-cycloalkyl, substituted or non-substituted C₁-C₈-halogenoalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulfanyl, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-halogenoalkoxycarbonyl having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylsulfanyl, substituted or non-substituted benzylamino, substituted or non-substituted phenoxy, substituted or non-substituted phenylsulfanyl, substituted or non-substituted phenylamino, a substituted or non-substituted or a 4-, 5-, 6- or 7-membered heterocycle comprising up to 4 heteroatoms selected in the list consisting of N, O, S,
when L² and Q² form together a substituted or non-substituted, 4-, 5-, 6- or 7-membered heterocycle comprising up to 4 heteroatoms selected in the list consisting of N, O, S, resulting heterocycles are non-aromatic.

10. A compound of formula (I) according to claim 1 wherein R^{d} to Rⁱ independently represent a hydrogen atom, a halogen atom, a nitro group, a cyano group, a hydroxy group, an amino group, a sulfanyl group, a formyl group, a formyloxy group, a formylamino group, substituted or non-substituted (hydroxyimino)-C₁-C₆-alkyl group, substituted or non-substituted C₁-C₈-alkyl, tri(C₁-C₈-alkyl)silyl, substituted or non-substituted tri(C₁-C₈-alkyl)silyl-C₁-C₈-alkyl, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-alkynyloxy, substituted or non-substituted C₁-C₈-alkylsulfanyl, substituted or non-substituted C₁-C₈-halogenoalkylsulfanyl having 1 to 5 halogen atoms, substituted or non-substituted C₁-C₈-alkylcarbonyl, substituted or non-substituted C₁-C₈-halogenoalkylcarbonyl having 1 to 5 halogen atoms, substituted or non-substituted C₁-C₈-alkoxycarbonyl, substituted or non-substituted C₁-C₈-halogenoalkoxycarbonyl having 1 to 5 halogen atoms, substituted or non-substituted C₁-C₈-alkylcarbonyloxy, substituted or non-substituted C₁-C₈-halogenoalkylcarbonyloxy having 1 to 5 halogen atoms, substituted or non-substituted C₁-C₈-alkylcarbonylamino, substituted or non-substituted C₁-C₈-halogenoalkylcarbonylamino having 1 to 5 halogen atoms, substituted or non-substituted C₁-C₈-alkylaminocarbonyloxy, substituted or non-substituted di-C₁-C₈-alkylaminocarbonyloxy, substituted or non-substituted C₁-C₈-alkyloxycarbonyloxy, substituted or non-substituted C₁-C₈-alkylsulphenyl, substituted or non-substituted C₁-C₈-halogenoalkylsulphenyl having 1 to 5 halogen atoms, substituted or non-substituted (C₁-C₆-alkoxyimino)-C₁-C₆-alkyl, substituted or non-substituted (C₁-C₆-alkenyloxyimino)-C₁-C₆-alkyl, substituted or non-substituted (C₁-C₆-alkynyloxyimino)-C₁-C₆-alkyl, (2-oxopyrrolidin-1-yl) C₁-C₈-alkyl, (2-oxopyrrolidin-1-yl) C₁-C₈-halogenoalkyl having 1 to 5 halogen atoms, (2-oxopiperidin-1-yl) C₁-C₈-alkyl, (2-oxopiperidin-1-yl) C₁-C₈-halogenoalkyl having 1 to 5 halogen atoms, (2-oxoazepan-1-yl) C₁-C₈-alkyl, (2-oxoazepan-1-yl) C₁-C₈-halogenoalkyl having 1 to 5 halogen atoms, substituted or non-substituted (benzyloxyimino)-C₁-C₆-alkyl, or a 4-, 5-, 6- or 7-membered heterocycle comprising up to 4 heteroatoms selected in the list consisting of N, O, S.

11. A compounds of formula (II) useful as intermediate compound or material for the preparation of a compound of formula (I) according to claim 1 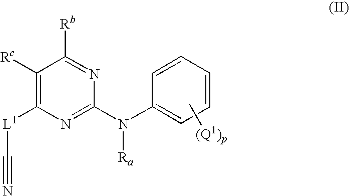
wherein
Q¹ independently represents a halogen atom, a nitro group, a hydroxy group, a cyano group, an amino group, a sulfanyl group, a pentafluoro-λ⁶-sulfanyl group, a formyl group, a formyloxy group, a formylamino group, a carbamoyl group, a N-hydroxycarbamoyl group, a carbamate group, a (hydroxyimino)-C₁-C₆-alkyl group, a C₁-C₈-alkyl, a substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a 2-oxopyrrolidin-1-yl, substituted or non-substituted (benzyloxyimino)-C₁-C₆-alkyl, substituted or non-substituted C₁-C₈-alkoxyalkyl, substituted or non-substituted C₁-C₈-halogenoalkoxyalkyl having 1 to 5 halogen atoms, substituted or non-substituted benzyloxy, substituted or non-substituted benzylsulfanyl, substituted or non-substituted benzylamino, substituted or non-substituted phenoxy, substituted or non-substituted phenylsulfanyl, or substituted or non-substituted phenylamino;
p represents 0, 1, 2, 3, 4 or 5;
R^{a} represents a hydrogen atom, a cyano group, a formyl group, a formyloxy group, a C₁-C₈-alkoxycarbonyl, a C₁-C₈-halogenoalkoxycarbonyl having 1 to 5 halogen atoms, a C₁-C₈-alkylcarbonyl, a C₁-C₈-halogenoalkylcarbonyl having 1 to 5 halogen atoms, a C₁-C₈-alkylsulphonyl, a C₁-C₈-halogenoalkylsulphonyl having 1 to 5 halogen atoms, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₁-C₈-alkoxyalkyl, a C₁-C₈-halogenoalkoxyalkyl having 1 to 5 halogen atoms,
R^{b} and R^{c} independently represent a hydrogen atom, a halogen atom, a cyano, a C₁-C₈-alkyl, a C₁-C₈-cycloalkyl, a C₁-C₈-halogenoalkyl having 1 to 5 halogen atoms, a C₁-C₈-halogenocycloalkyl having 1 to 5 halogen atoms; and
L¹ represents a substituted or non substituted pyridyl moiety.

12. A fungicide composition comprising, as an active ingredient, an effective amount of a compound of formula (I) according to claim 1 and an agriculturally acceptable support, carrier or filler.

13. A method for controlling phytopathogenic fungi of crops, characterized in that an agronomically effective and substantially non-phytotoxic quantity of a compound according to claim 1 is applied to the soil where plants grow or are capable of growing, to the leaves and/or the fruit of plants or to the seeds of plants.

14. A method for controlling phytopathogenic fungi of crops, characterized in that an agronomically effective and substantially non-phytotoxic quantity of a composition according claim 12 is applied to the soil where plants grow or are capable of growing, to the leaves and/or the fruit of plants or to the seeds of plants.
